(19) United States
(12) Patent Application Publication
(10) Pub. No.: US 2026/0069110 A1
(43) Pub. Date: Mar. 12, 2026

(54) MEDICAL SUPPORT DEVICE, MEDICAL SUPPORT SYSTEM, AND METHOD OF OPERATING MEDICAL SUPPORT DEVICE

(71) Applicant: OLYMPUS MEDICAL SYSTEMS CORP., Tokyo (JP)

(72) Inventors: Haruhiko SAKAYORI, Kawasaki-shi (JP); Ryosuke SAKAI, Tokyo (JP); Megumi NAGASAWA, Tokyo (JP); Taishi KAKEMIZU, Tokyo (JP)

(73) Assignee: OLYMPUS MEDICAL SYSTEMS CORP., Tokyo (JP)

(21) Appl. No.: 19/392,690

(22) Filed: Nov. 18, 2025

Related U.S. Application Data
(63) Continuation of application No. PCT/JP2023/022381, filed on Jun. 16, 2023.

(51) Int. Cl. A61B 1/00; A61B 90/00

(52) U.S. Cl. CPC A61B 1/00009; A61B 1/00006; A61B 1/0002; A61B 1/00045; A61B 90/06; A61B 2090/062

(57) ABSTRACT

A medical support device includes a processor including hardware. The processor is configured to: acquire a first image data group and examination information, determine, based on observation order information, whether a distal end portion of an insertion portion provided in an endoscope reaches a final arrival site set in an observation order, discriminate, based on determining that the distal end portion reaches the final arrival site, between first image data captured before the distal end portion reaches the final arrival site and a second image data group captured after the distal end portion reaches the final arrival, and generate, based on a determination result obtained by discriminating between the first image data and the second image data group and on the examination information, related information including time information related to movement of the endoscope in the second image data group.

### CROSS REFERENCES TO RELATED APPLICATIONS

This application is a continuation of International Application No. PCT/JP2023/022381, filed on Jun. 16, 2023, the entire contents of which are incorporated herein by reference.

### BACKGROUND

### 1. Technical Field

The present disclosure relates to a medical support device, a medical support system, and a method of operating a medical support device that perform medical support including an endoscope examination report.

### 2. Related Art

In the related art, in an endoscope, disclosed is a technology of generating a distal end marker array image in which a plurality of distal end markers representing a position and an angle of a distal end of an insertion portion of the endoscope at predetermined time intervals are connected and arranged based on position information and angle information at the distal end of the insertion portion of the endoscope, which are acquired at predetermined time intervals (for example, refer to JP 2017-47009 A). In this technique, the distal end marker array image at predetermined time intervals is superimposed and displayed at a position corresponding to a schema diagram of a target organ, thereby reducing labor and time required for creation of the endoscope report.

### SUMMARY

In some embodiments, a medical support device includes a processor including hardware, the processor being configured to: acquire a first image data group and examination information, the first image data group being generated by allowing an endoscope to continuously capture images of a plurality of observed regions in a subject, the examination information including detection result information obtained by detecting each of the plurality of observed regions based on the image data group, determine, based on observation order information in which an observation order of a preset organ and a preset site is set, the first image data group, and the examination information, whether a distal end portion of an insertion portion provided in the endoscope reaches a final arrival site set in the observation order, discriminate, based on determining that the distal end portion reaches the final arrival site, between first image data captured before the distal end portion reaches the final arrival site and a second image data group captured after the distal end portion reaches the final arrival, and generate, based on a determination result obtained by discriminating between the first image data and the second image data group and on the examination information, related information including time information related to movement of the endoscope in the subject in the second image data group.

In some embodiments, a medical support system includes: an endoscope; a control device configured to control the endoscope; an image diagnosis device; and a medical support device. The endoscope includes: an insertion portion configured to be inserted into a subject; and a distal end portion provided at a distal end of the insertion portion, the distal end portion being configured to sequentially generate a first image data group by continuously capturing images of a plurality of observed regions preset in the subject and to output the first image data group to the control device, the image diagnosis device is configured to generate detection result information in which each of the plurality of observed regions is detected for the first image data group using the first image data group and a first inference model that has been pretrained on features of an organ and a site and to output the detection result information to the control device, the medical support device includes a first processor comprising hardware, and the first processor is configured to: acquire, from the control device, the first image data group and examination information including the detection result information generated by the image diagnosis device, discriminate, based on the first image data group and the examination information, between first image data captured during observation of a predetermined observed region and a second image data group captured after the observation of the predetermined observed region, and generate, based on a discrimination result obtained by discriminating between the first image data and the second image data group and on the examination information, related information related to movement of the endoscope in the subject in the second image data group.

In some embodiments, provided is a method of operating a medical support device. The method causes the medical support device to execute: acquiring an image data group and examination information, the image data group being generated by allowing an endoscope to continuously capture images of a plurality of observed regions in a subject, the examination information including detection result information obtained by detecting each of the plurality of observed regions based on the image data group, discriminating, based on the image data group and the examination information, between first image data captured during observation of a predetermined observed region and a second image data group captured after the observation of the predetermined observed region, and generating, based on a discrimination result obtained by discriminating between the first image data and the second image data group and on the examination information, related information related to movement of the endoscope in the subject in the second image data group.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an overall configuration of a medical support system according to an embodiment;

FIG. 2 is a block diagram illustrating a functional configuration of a main part of an endoscope and a control device according to the embodiment;

FIG. 3 is a block diagram illustrating a functional configuration of an image diagnosis device according to the embodiment;

FIG. 4 is a block diagram illustrating a functional configuration of a medical support device according to the embodiment;

FIG. 5 is a flowchart illustrating an outline of processing executed by the medical support system according to the embodiment;

FIG. 6 is a diagram illustrating an example of an observation order of organs/sites and a list of report items;

FIG. 7 is a diagram illustrating another example of the observation order of organs/sites and the list of report items;

FIG. 8 is a flowchart illustrating an outline of endoscope imaging processing in FIG. 5;

FIG. 9 is a flowchart illustrating an outline of report creation processing in FIG. 5;

FIG. 10 is a diagram schematically illustrating an outline when a discrimination unit included in the medical support device according to the embodiment discriminates an image data group into first image data and a second image data group;

FIG. 11 is a diagram illustrating an example of examination information acquired by an acquisition unit included in the medical support device according to the embodiment;

FIG. 12 is a diagram schematically illustrating a discrimination method in which discrimination is performed by the discrimination unit included in the medical support device according to the embodiment;

FIG. 13 is a diagram illustrating another example of the examination information acquired by the acquisition unit included in the medical support device according to the embodiment;

FIG. 14 is a diagram schematically illustrating a discrimination method in which discrimination is performed by the discrimination unit included in the medical support device according to the embodiment;

FIG. 15 is a diagram illustrating another example of the examination information acquired by the acquisition unit included in the medical support device according to the embodiment;

FIG. 16 is a diagram schematically illustrating a discrimination method in which discrimination is performed by the discrimination unit included in the medical support device according to the embodiment;

FIG. 17 is a diagram schematically illustrating a method of calculating an insertion time calculated by a calculation unit included in the medical support device according to the embodiment;

FIG. 18 is a diagram illustrating an example of an examination report created by a report creation unit included in the medical support device according to the embodiment; and

FIG. 19 is a flowchart illustrating an outline of unexamined processing in FIG. 5.

### DETAILED DESCRIPTION

Hereinafter, modes for carrying out the present disclosure will be described in detail with reference to the drawings. Note that the present disclosure is not limited to the following embodiments. In addition, each drawing referred to in the following description merely schematically illustrates a shape, a size, and a positional relationship to an extent that a content of the present disclosure can be understood. That is, the present disclosure is not limited only to the shape, the size, and the positional relationship illustrated in each drawing. Furthermore, in the description of the drawings, the same portions will be denoted by the same reference numerals.

### Overall Configuration of Medical Support System

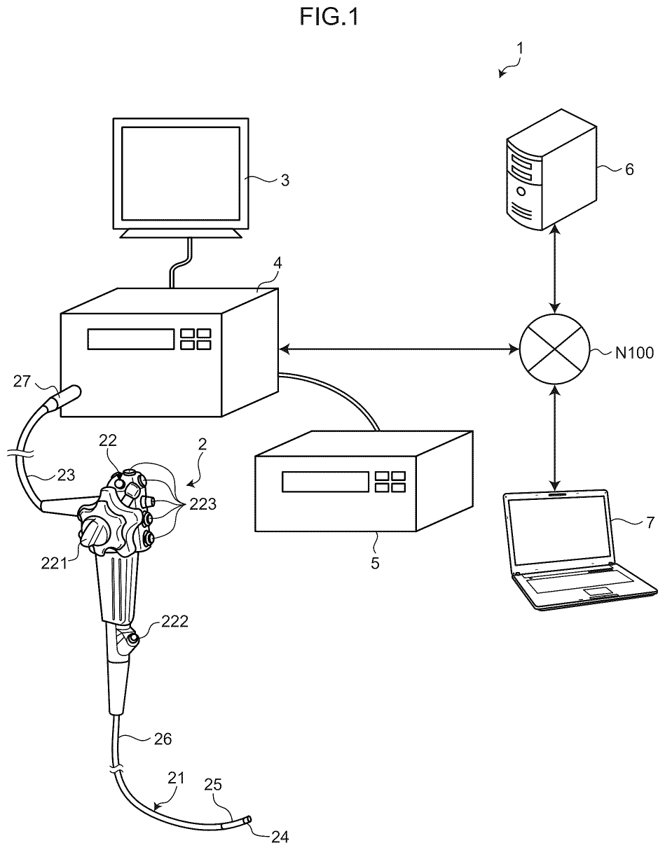

FIG. 1 is a diagram illustrating an overall configuration of a medical support system according to an embodiment. A medical support system 1 illustrated in FIG. 1 continuously captures images of the inside of a subject by inserting an insertion portion 21 of an endoscope 2 from the mouth of the subject such as a person or an animal to the esophagus or from the anus of the subject to the large intestine, and displays a display image of image data group including the captured images on a display device 3 in chronological order.

Furthermore, the medical support system 1 detects (recognizes or estimates) a characteristic region including an organ, a site, an insertion speed of the insertion portion 21, the polyp, cancer, or the like of a plurality of observed regions appearing in the image data based on the image data group input from a control device 4 by an image diagnosis device 5 (CAD: Computer Aided Detection) and an inference model (learned model) learned by using learning data obtained by imaging an organ, a site, a lesion, or the like of the subject in advance, and outputs the detection result to the control device 4. An operator such as a doctor observes and treats the subject while confirming the display image displayed on the display device 3 and the detection result (estimation result) of the image diagnosis device 5.

In addition, the medical support system 1 acquires an image data group input from the control device 4 by a medical support device 6 via a network N100 and a piece of examination information including the detection result (estimation result) of the image diagnosis device 5, and records the acquired image data and the examination information in association with each other. Then, the medical support system 1 outputs various data in response to a request from a laptop personal computer 7 of the operator or the like via the network N100.

Furthermore, in the medical support system 1, report information such as an examination report of the subject by the operator or the like is created in the laptop personal computer 7, and the report information is output to and recorded in the medical support device 6. Thereafter, the operator diagnoses the subject and conducts a conference based on the report information, diagnosis results of biological cells, which are acquired by biopsy at the time of observing the subject, and the like.

As illustrated in FIG. 1, the medical support system 1 includes the endoscope 2, the display device 3, the control device 4, the image diagnosis device 5, and the medical support device 6.

### Configuration of Endoscope

The endoscope 2 continuously generates image data (RAW data) by capturing an image of the inside of the subject, and sequentially outputs the image data to the control device 4. As illustrated in FIG. 1, the endoscope 2 includes the insertion portion 21, an operating portion 22, and a universal cord 23.

At least a part of the insertion portion 21 has flexibility and is inserted into the subject. As illustrated in FIG. 1, the insertion portion 21 includes a distal end portion 24 provided at a distal end of the insertion portion 21, a curved portion 25 connected to a proximal end side (operating portion 22 side) of the distal end portion 24 and configured to be bendable, and an elongated flexible tube portion 26 connected to a proximal end side of the curved portion 25 and formed to have flexibility.

The operating portion 22 is connected to a proximal end portion of the insertion portion 21. The operating portion 22 receives various operations on the endoscope 2. As illustrated in FIG. 1, the operating portion 22 is provided with a bending knob 221, an insertion port 222, and a plurality of operating portions 223.

The bending knob 221 is configured to be rotatable in response to a user operation by a user such as an operator. The bending knob 221 turns to operate a bending mechanism (not illustrated) such as a metal or resin wire disposed in the insertion portion 21. As a result, the curved portion 25 is curved.

The insertion port 222 communicates with a treatment instrument channel (not illustrated) which is a conduit extending from the distal end of the insertion portion 21, and is an insertion port for inserting a treatment instrument or the like into the treatment instrument channel from the outside of the endoscope 2.

The plurality of operating portions 223 includes buttons and the like that receive various operations by a user such as an operator, and outputs operation signals corresponding to the various operations to the control device 4 via the universal cord 23. Examples of the various operations can include a release operation of instructing a still image by the endoscope 2, an operation of switching an observation mode of the endoscope 2 to a normal light observation mode or a special observation mode, and the like.

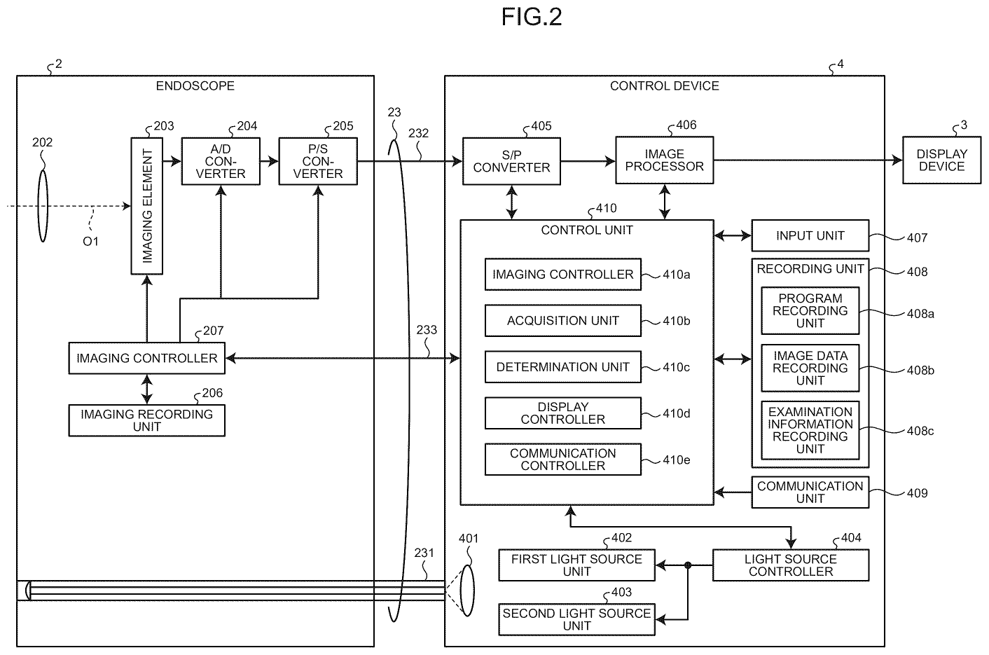

The universal cord 23 is a cord formed to extend from the operating portion 22 in a direction different from the extending direction of the insertion portion 21 and provided with a light guide 231 (refer to FIG. 2) including an optical fiber or the like, a first signal line 232 (refer to FIG. 2) for transmission of the image data described above, a second signal line 233 (refer to FIG. 2) for transmission of the operation signal described above, and the like. As illustrated in FIG. 1, a first connector portion 27 is provided at the proximal end of the universal cord 23. The first connector portion 27 is detachably connected to the control device 4.

The display device 3 includes a display monitor such as liquid crystal or organic electro luminescence (EL), and displays, under the control of the control device 4, a display image based on image data subjected to image processing in the control device 4 and various types of information regarding the endoscope 2.

The control device 4 is implemented by using a processor, which is a processing device having hardware such as a graphics processing unit (GPU), a field programmable gate array (FPGA), or a central processing unit (CPU), and a memory, which is a temporary storage area used by the processor. The control device 4 integrally controls the operation of each unit of the endoscope 2 according to a program recorded in the memory.

The image diagnosis device 5 (CAD) detects (estimates) an organ, a site, an insertion speed of the insertion portion 21, and a characteristic region (an abnormal region or lesion candidate region) including a polyp, cancer, or the like, which appear in image data, by using an image data group input from the control device 4 and a learned model prelearned by learning data, and outputs examination information in which this detection result and a detection time are associated with each other to the control device 4. The image diagnosis device 5 is implemented by using a processor, which is a processing device having hardware such as a GPU, an FPGA, or a CPU, and a memory, which is a temporary storage area used by the processor.

The medical support device 6 sequentially records various data input from the control device 4 and a recognition result of the image diagnosis device 5, and transmits data in response to a request from the laptop personal computer 7. The medical support device 6 is implemented by using a processor, which is a processing device having hardware such as a GPU, an FPGA, or a CPU, and a memory, which is a temporary storage area used by the processor. Furthermore, the medical support device 6 is configured using a hard disk drive (HDD), a solid state drive (SSD), and the like.

### Functional Configuration of Main Part of Medical Support System

Next, a functional configuration of a main part of the above-described medical support system 1 will be described.

FIG. 2 is a block diagram illustrating a functional configuration of a main part of the endoscope 2 and the control device 4. Hereinafter, the endoscope 2 and the control device 4 will be described in this order, and then the image diagnosis device 5 and the medical support device 6 will be described.

### Functional Configuration of Endoscope

First, a configuration of the endoscope 2 will be described.

As illustrated in FIG. 2, the endoscope 2 includes an illumination optical system 201, an imaging optical system 202, an imaging element 203, an A/D converter 204, a P/S converter 205, an imaging recording unit 206, and an imaging controller 207. Here, each of the illumination optical system 201, the imaging optical system 202, the imaging element 203, the A/D converter 204, the P/S converter 205, the imaging recording unit 206, and the imaging controller 207 is disposed in the distal end portion 24.

The illumination optical system 201 includes one or a plurality of lenses and the like, and irradiates the subject with illumination light supplied from the light guide 231.

The imaging optical system 202 includes one or a plurality of lenses and the like, and condenses reflected light reflected from a subject, return light from the subject, and light such as fluorescence emitted from the subject to form a subject image on a light receiving surface of the imaging element 203. In addition, the imaging optical system 202 can change a focal length and an angle of view by moving the lens in an optical axis direction O1.

The imaging element 203 is configured using a charge coupled device (CCD) or a complementary metal oxide semiconductor (CMOS) image sensor in which any one of color filters constituting a Bayer array (RGGB) is arranged in each of a plurality of pixels arranged in a two-dimensional matrix. Under the control of the imaging controller 207, the imaging element 203 receives the subject image formed by the imaging optical system 202 and performs photoelectric conversion to generate a captured image (an analog signal). Note that, in the present embodiment, the imaging element 203 may be formed to be integrated with an image sensor and a TOF sensor that acquires subject distance information (hereinafter, described as depth map information) by a TOF (Time Of Flight) method. The depth map information is information in which the subject distance from the position of the imaging element 203 (the position of the distal end portion 24) to the corresponding position on an observation target corresponding to the pixel position in the captured image is detected for each pixel position. Note that the configuration for generation of the depth map information is not limited to the above-described TOF sensor, and an image sensor or the like including a phase difference sensor may be adopted. Hereinafter, the depth map information and the captured image will be collectively referred to as image data. The imaging element 203 outputs the image data to the A/D converter 204.

The A/D converter 204 is configured using an A/D conversion circuit or the like. Under the control of the imaging controller 207, the A/D converter 204 performs A/D conversion processing on analog image data input from the imaging element 203, and outputs the digital image data to the P/S converter 205.

The P/S converter 205 is configured using a P/S conversion circuit or the like, performs parallel/serial conversion of digital image data input from the A/D converter 204 under the control of the imaging controller 207, and outputs the digital image data to the control device 4 via the first signal line 232.

Note that an E/O converter that converts image data into an optical signal may be provided instead of the P/S converter 205, and the image data may be configured to be output to the control device 4 by the optical signal. In addition, image data may be configured to be transmitted to the control device 4 by wireless communication such as Wireless Fidelity (Wi-Fi) (registered trademark).

The imaging recording unit 206 is formed by a nonvolatile memory or a volatile memory, and records various types of information regarding the endoscope 2 (for example, pixel information of the imaging element 203). Furthermore, the imaging recording unit 206 records various setting data and control parameters transmitted from the control device 4 via the second signal line 233.

The imaging controller 207 is implemented by using a timing generator (TG), a processor, which is a processing device having hardware such as a CPU, and a memory, which is a temporary storage area used by the processor. The imaging controller 207 controls the operation of each of the imaging element 203, the A/D converter 204, and the P/S converter 205 based on the setting data received from the control device 4 via the second signal line 233.

### Configuration of Control Device

Next, a configuration of the control device 4 will be described.

As illustrated in FIG. 2, the control device 4 includes a condenser lens 401, a first light source unit 402, a second light source unit 403, a light source controller 404, an S/P converter 405, an image processor 406, an input unit 407, a recording unit 408, a communication unit 409, and a control unit 410.

The condenser lens 401 condenses light emitted by each of the first light source unit 402 and the second light source unit 403 and emits the light to the light guide 231.

Under the control of the light source controller 404, the first light source unit 402 emits white light (normal light), which is visible light, to supply the white light to the light guide 231 as illumination light. The first light source unit 402 is configured using a collimating lens, a white light emitting diode (LED) lamp, a drive driver, and the like. Note that, as the first light source unit 402, white light of visible light may be supplied by simultaneously emitting light using a red LED lamp, a green LED lamp, and a blue LED lamp. The first light source unit 402 may include a halogen lamp, a xenon lamp, or the like.

The second light source unit 403 emits special light having a predetermined wavelength band under the control of the light source controller 404 to supply the special light to the light guide 231 as illumination light. Here, the special light is light used for narrowband imaging (NBI) with narrowband light including 390 to 445 nm and 530 to 550 nm. Of course, in addition to the narrowband light, amber light (600 nm and 630 nm) used for red dichromatic imaging (RDI) may be used as the special light.

The light source controller 404 is implemented by using a processor having hardware such as an FPGA or a CPU, and a memory, which is a temporary storage area used by the processor. The light source controller 404 controls a light emission timing, a light emission time, and the like of each of the first light source unit 402 and the second light source unit 403 based on control data input from the control unit 410.

Under the control of the control unit 410, the S/P converter 405 performs serial/parallel conversion on image data received from the endoscope 2 via the first signal line 232, and outputs the image data to the image processor 406. Note that, in a case where the endoscope 2 outputs the image data as an optical signal, an O/E converter that converts the optical signal into an electrical signal may be provided instead of the S/P converter 405. In addition, in a case where the endoscope 2 transmits image data by wireless communication, a communication module capable of receiving a wireless signal may be provided instead of the S/P converter 405.

The image processor 406 is implemented by using a processor having hardware such as a GPU or an FPGA and a memory, which is a temporary storage area used by the processor. Under the control of the control unit 410, the image processor 406 performs predetermined image processing on image data of the parallel data input from the S/P converter 405, and outputs the processed image data to the display device 3. Here, examples of the predetermined image processing include demosaic processing, white balance processing, gain adjustment processing, γ correction processing, format conversion processing, and the like.

The input unit 407 is configured using a mouse, a foot switch, a keyboard, a button, a switch, a touch panel, and the like, receives a user operation from a user such as an operator, and outputs an operation signal corresponding to the user operation to the control unit 410.

The recording unit 408 is configured using a recording medium such as a volatile memory, a nonvolatile memory, a solid state drive (SSD), a hard disk drive (HDD), or a memory card. Then, the recording unit 408 records data including various parameters and the like necessary for the operation of the control device 4 and the endoscope 2. Furthermore, the recording unit 408 includes a program recording unit 408a that records various programs for the operation of the endoscope 2 and the control device 4, an image data recording unit 408b that records image data, and an examination information recording unit 408c that records examination information.

Under the control of the control unit 410, the image data recording unit 408b records an image data group and still image data which are generated by allowing the endoscope 2 to continuously capture images of a plurality of observed regions on the subject in association with patient information or the like.

The examination information recording unit 408c records the examination information. Here, the examination information includes detection result information input from the image diagnosis device 5, detection time of the detection result, still image data, imaging time at which the still image data is captured, speed information in which insertion speed information regarding the insertion speed of the distal end portion 24 of the endoscope 2 and measurement time at which the insertion speed of the distal end portion 24 of the endoscope 2 is measured are associated with each other, and patient information. The still image data is image data captured by the imaging element 203 when imaging is instructed by a release signal input from the operating portion 22, and is image data having a higher resolution than that of a live view image sequentially generated by the imaging element 203. In addition, the insertion speed information is a binarized value in which the movement of the distal end portion 24 is “faster” or “slower” than a predetermined threshold. Of course, it is not necessary to perform binarization, and multi-stage values such as “fast”, “normal”, and “slow” may be used by providing a plurality of values.

Under the control of the control unit 410, the communication unit 409 transmits various types of information to the medical support device 6 via the image diagnosis device 5 and the network N100, receives various types of information from the image diagnosis device 5 and the medical support device 6, and outputs the information to the control unit 410. Specifically, under the control of the control unit 410, the communication unit 409 transmits a temporally continuous image data group on which the image processor 406 has performed image processing to the image diagnosis device 5, receives a detection result including an organ, a site, a special region, an insertion speed of the endoscope 2, and the like detected in the image diagnosis device 5, and outputs the detection result to the control unit 410. In addition, under the control of the control unit 410, the communication unit 409 transmits examination information including the image data group, the still image data, and the detection result of the image diagnosis device 5 to the medical support device 6, and receives patient information of a subject, observation information indicating an observation order of an observation place at the time of the examination of the subject, and the like from the medical support device 6. The communication unit 409 is configured using a communication module or the like.

The control unit 410 corresponds to a second processor according to the present disclosure. The control unit 410 is implemented by using a second processor, which is a processing device having hardware such as an FPGA or a CPU, and a memory, which is a temporary storage area used by the second processor. Then, the control unit 410 integrally controls each unit constituting the endoscope 2 and the control device 4. The control unit 410 includes an imaging controller 410a, an acquisition unit 410b, a determination unit 410c, a display controller 410d, and a communication controller 410e.

The imaging controller 410a controls imaging of the endoscope 2. The imaging controller 410a causes the endoscope 2 to continuously capture images to generate temporally continuous image data groups. Furthermore, in a case where a release signal is input from the operating portion 22, the imaging controller 410a causes the imaging element 203 to capture still image data.

The acquisition unit 410b acquires image data from the endoscope 2 via the S/P converter 405 or the image processor 406 and records the image data in the image data recording unit 408b. The acquisition unit 410b acquires detection result information from the image diagnosis device 5 via the communication unit 409 and records the detection result information in the examination information recording unit 408c.

The determination unit 410c determines whether the distal end portion 24 of the endoscope 2 has reached a final arrival site based on observation order information of the final arrival site set in the observation order of report creation and the site of the subject, which is detected by the image diagnosis device 5. The determination unit 410c determines whether the final arrival site has not been captured. Specifically, the determination unit 410c determines whether the still image data obtained by capturing an image of the final arrival site of the organ/site set in the observation order (insertion order) in the report item in the examination information recording unit 408c is recorded in the image data recording unit 408b.

When the determination unit 410c determines that the distal end portion 24 of the endoscope 2 has reached the final arrival site, the display controller 410d causes the display device 3 to display information indicating that the distal end portion 24 of the endoscope 2 has reached the final arrival site in the subject. In addition, in a case where the determination unit 410c determines that the image of the final arrival site has not been captured, the display controller 410d causes the display device 3 to display a warning indicating that the image of the final arrival site has not been captured.

The communication controller 410e causes the communication unit 409 to transmit various types of information to the medical support device 6 and causes the communication unit 409 to receive various types of information from the medical support device 6 via the network N100.

### Functional Configuration of Image Diagnosis Device

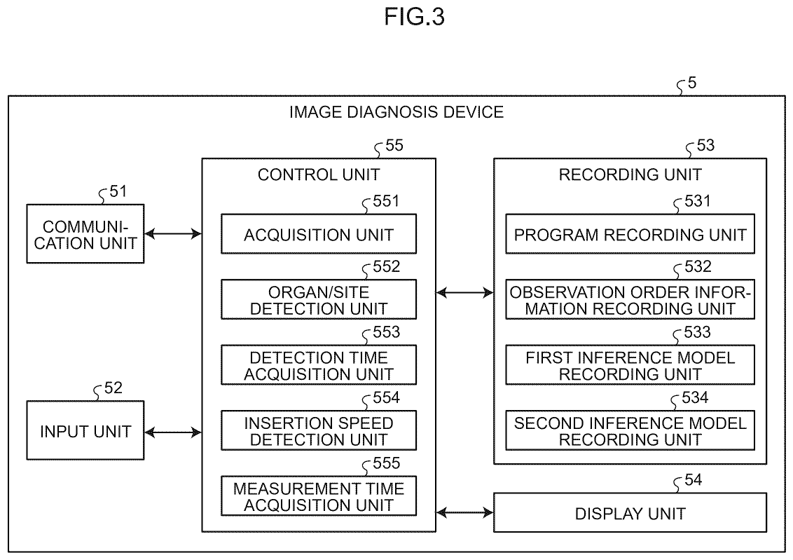

Next, a functional configuration of the image diagnosis device 5 will be described. FIG. 3 is a block diagram illustrating a functional configuration of the image diagnosis device 5. The image diagnosis device 5 illustrated in FIG. 3 includes a communication unit 51, an input unit 52, a recording unit 53, a display unit 54, and a control unit 55.

Under the control of the control unit 55, the communication unit 51 receives an image data group generated by the endoscope 2 and organ/site order information indicating the observation order of the organ/site in the subject as a patient from the control device 4 via the network N100, and outputs the image data group and the organ/site order information to the control unit 55. In addition, under the control of the control unit 55, the communication unit 51 outputs, to the control device 4, examination information including a detection result of detecting an organ or the like of the subject input from the control unit 55 and a detection time at which the detection result is detected via the network N100. The communication unit 51 is configured using a communication module or the like.

The input unit 52 includes a button, a switch, a touch panel, and the like, receives various inputs, and outputs the inputs to the control unit 55.

The recording unit 53 is configured using a recording medium such as a volatile memory, a nonvolatile memory, an SSD, an HDD, or a memory card. The recording unit 53 records data including various parameters and the like necessary for the operation of the image diagnosis device 5. In addition, the recording unit 53 includes a program recording unit 531 that records various programs for operating the image diagnosis device 5, an observation order recording unit 532, a first inference model recording unit 533, and a second inference model recording unit 534.

The observation order recording unit 532 records observation order information in which an observation order of a plurality of observation points such as an organ and a site preset is set at the time of observing the subject by the endoscope 2 input from the medical support device 6 to be described later.

The first inference model recording unit 533 records a first inference model used to detect an organ, a site, and a characteristic region based on image data. Here, the first inference model uses an input as image data of the endoscope 2 and outputs an organ, a site, and a characteristic region included in the image data as an output. This first inference model is, for example, a model generated by machine learning using artificial intelligence (AI). Specifically, the first inference model is a model such as deep learning that performs learning using teacher data obtained by adding an organ, a site, and a characteristic region such as a lesion of a subject to image data obtained by capturing an image of the subject and outputs the organ, the site, and the characteristic region included in the image data.

The second inference model recording unit 534 records a second inference model used to detect the insertion speed of the distal end portion 24 of the endoscope 2. Here, the second inference model uses the image data of the endoscope 2 as an input and outputs insertion speed information regarding the insertion speed of the distal end portion 24 of the endoscope 2 as an output. The second inference model is a model such as deep learning that performs learning using training data obtained by adding an insertion speed and a movement distance of the distal end portion 24 of the endoscope 2 to image data obtained by capturing an image of the subject and outputs the insertion speed of the distal end portion 24 of the endoscope 2 from the image data.

The display unit 54 displays various types of information regarding the image diagnosis device 5 under the control of the control unit 55. The display unit 54 is configured using a liquid crystal display, an organic EL display, or the like.

The control unit 55 is implemented by using a processor having hardware such as an FPGA or a CPU and a memory, which is a temporary storage area used by the processor. Then, the control unit 55 integrally controls each unit constituting the medical support device 6. The control unit 55 includes an acquisition unit 551, an organ/site detection unit 552, a detection time acquisition unit 553, an insertion speed detection unit 554, and a measurement time acquisition unit 555.

The acquisition unit 551 acquires image data sequentially generated by the endoscope 2 from the control device 4 via the communication unit 51 in chronological order.

The organ/site detection unit 552 detects an organ/site and a characteristic region of the subject, which are included in the image data, based on the image data sequentially acquired by the acquisition unit 551 and the first inference model recorded in the first inference model recording unit 533.

The detection time acquisition unit 553 acquires a detection time when the organ/site detection unit 552 detects the organ/site and the characteristic region of the subject, which are included in the image data. For example, when the organ/site detection unit 552 detects the organ/site and the characteristic region of the subject, which are included in the image data, the detection time acquisition unit 553 acquires a time measured by TG (not illustrated) as the detection time.

The insertion speed detection unit 554 detects the insertion speed of the distal end portion 24 of the endoscope 2 based on the image data sequentially acquired by the acquisition unit 551 and the second inference model recorded in the second inference model recording unit 534.

The measurement time acquisition unit 555 acquires a measurement time when the insertion speed of the distal end portion 24 of the endoscope 2, which is detected by the insertion speed detection unit 554, is detected. For example, when detecting the insertion speed of the distal end portion 24 of the endoscope 2, which is detected by the insertion speed detection unit 554, the detection time acquisition unit 553 acquires a time measured by TG (not illustrated) as the measurement time.

### Functional Configuration of Medical Support Device

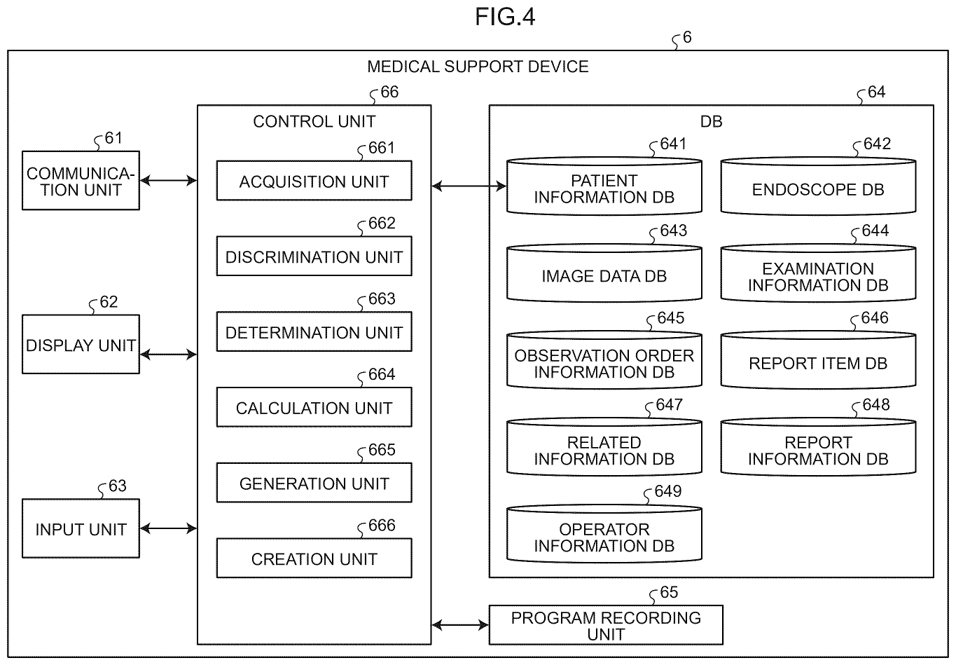

Next, a functional configuration of the medical support device 6 will be described. FIG. 4 is a block diagram illustrating a functional configuration of the medical support device 6. The medical support device 6 illustrated in FIG. 4 includes a communication unit 61, a display unit 62, an input unit 63, a database 64 (hereinafter, simply referred to as “DB 64”), a program recording unit 65, and a control unit 66.

Under the control of the control unit 66, the communication unit 61 transmits various types of information to the control device 4 and the image diagnosis device 5 via the network N100, and receives various types of information from the control device 4 and the image diagnosis device 5. The communication unit 61 is configured using a communication module.

The display unit 62 displays various types of information regarding the medical support device 6 under the control of the control unit 66. The display unit 62 is configured using a liquid crystal display, an organic EL display, or the like.

The input unit 63 receives various inputs and outputs the inputs to the control unit 66 in response to an operation from the outside. The input unit 63 includes a button, a keyboard, a switch, a touch panel, and the like.

The DB 64 records various types of information. The DB 64 is configured using an HDD, an SSD, and the like. The DB 64 includes a patient information DB 641, an endoscope DB 642, an image data DB 643, an examination information DB 644, an observation order information DB 645, a report item DB 646, a related information DB 647, a report information DB 648, and an operator information DB 649.

The patient information DB 641 records, for each patient, a patient ID for identifying the patient, a date of birth of the patient, a doctor in charge of the patient, an address, and allergy information of the patient.

The endoscope DB 642 records, for each endoscope 2, a type of the endoscope 2, an endoscope ID, a repair history of the endoscope 2, a maintenance status of the endoscope 2, a use history, and use schedule information.

The image data DB 643 records an image data group captured by the endoscope 2 in association with each patient ID or each operator ID.

The examination information DB 644 records examination information including detection result information obtained by detecting each of a plurality of observed regions based on an image data group detected by the image diagnosis device 5 in association with each patient ID or each operator ID.

The observation order information DB 645 records the observation order of the organ/site by the endoscope 2 set for each facility or each operator ID.

The report item DB 646 records a report item by the endoscope 2, which is set for each facility or each operator ID.

The related information DB 647 records related information related to the movement of the endoscope 2 in the subject in a second image data group generated by the medical support device 6 to be described later.

The report information DB 648 records, for each patient ID or each operator ID, report information created by an operator (an observer) using the endoscope 2 and report information automatically generated by the medical support device 6 in association with each other.

The operator information DB 649 records, for each operator ID, the name, the position, the affiliation, and the contact information in association with each other.

The program recording unit 65 is configured using a volatile memory, a nonvolatile memory, an SSD, and the like, and records various programs for operating the medical support device 6.

The control unit 66 corresponds to a first processor according to the present disclosure. The control unit 66 is implemented by using a first processor having hardware such as an FPGA or a CPU and a memory, which is a temporary storage area used by the first processor. Then, the control unit 66 integrally controls each unit constituting the medical support device 6. The control unit 66 includes an acquisition unit 661, a discrimination unit 662, a determination unit 663, a calculation unit 664, a generation unit 665, and a creation unit 666.

The acquisition unit 661 acquires image data group and the examination information from the control device 4 via the network N100.

Based on the image data group and the examination information acquired by the acquisition unit 661, the discrimination unit 662 discriminates between first image data captured at the time of observing a predetermined observed region and a second image data group captured after observing the predetermined observed region.

The calculation unit 664 calculates an insertion time until the distal end portion 24 of the endoscope 2 reaches the final arrival site based on an arrival time of the final arrival site discriminated by the discrimination unit 662 and the examination information.

The generation unit 665 generates related information including the insertion time until the distal end portion 24 of the endoscope 2 reaches the final arrival site, which is calculated by the calculation unit 664, the insertion start time included in the examination information, and the measurement time of the endoscope 2 based on an extracorporeal time detected as the outside of the body by the image diagnosis device 5, and records the generated related information in the related information DB 647.

The creation unit 666 creates report information and records the report information in the report information DB 648 based on operation information from the input unit 63 or the laptop personal computer 7, the image data group and the examination information acquired by the acquisition unit 661, the patient information, and the related information generated by the generation unit 665.

### Processing of Medical Support System

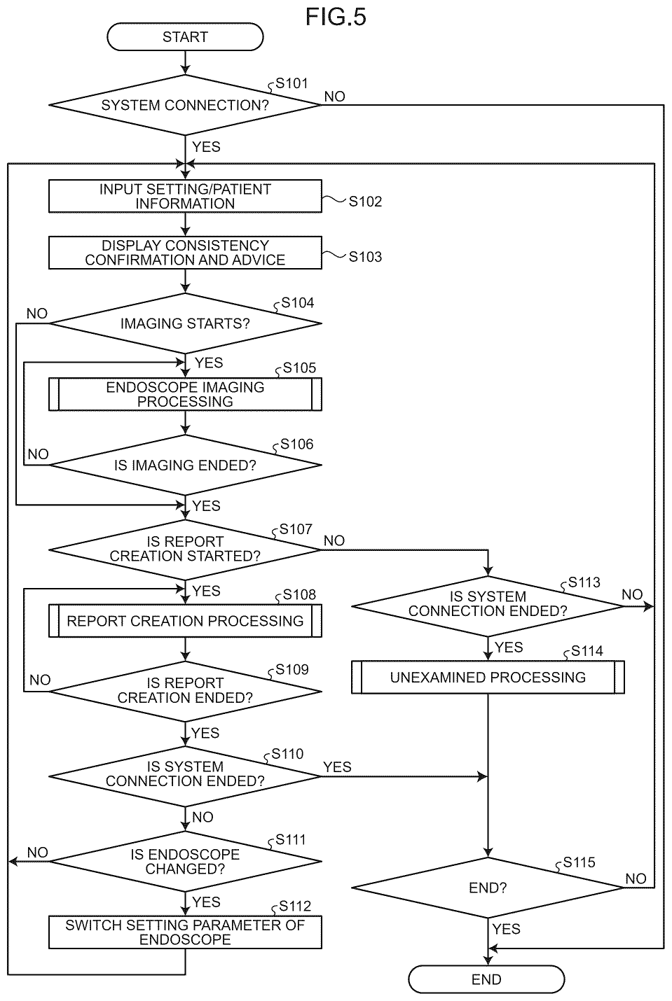

Next, an outline of processing executed by the medical support system 1 will be described. FIG. 5 is a flowchart illustrating an outline of processing executed by the medical support system 1.

As illustrated in FIG. 5, first, the control unit 66 determines whether the endoscope 2 is connected to the medical support system 1 by connecting the endoscope 2 to the control device 4 via the network N100 (step S101). Specifically, the control unit 66 determines whether an endoscope ID for identifying the endoscope 2, which is input from the control device 4 via the network N100, is input, and determines that the endoscope 2 is connected to the medical support system 1 in a case where the endoscope ID for identifying the endoscope 2 is input from the control device 4.

In a case where the control unit 66 determines that the endoscope 2 is connected to the medical support system 1 by connecting the endoscope 2 to the control device 4 (step S101: Yes), the medical support system 1 proceeds to step S102. On the other hand, in a case where the control unit 66 determines that the endoscope 2 is not connected to the control device 4 and the endoscope 2 is not connected to the medical support system 1 (step S101: No), the medical support system 1 ends the present processing.

Subsequently, the control unit 66 is configured to receive setting of the observation order of an organ/site of a subject, which is input from the laptop personal computer 7 or the control device 4, and then record the setting in the observation order information DB 645, and to receive an input of the patient name, and the like and then record the input in the patient information DB 641 (step S102). Specifically, based on an operation content input from the laptop personal computer 7 or the like of the operator, the control unit 66 receives input of various types of information such as the name of a patient (subject), a patient ID (subject ID) of the subject, and the date of birth of the patient (subject), a type of the endoscope 2 used for examination of the subject (subject), an operator ID for identifying an attending doctor, a type of a report item, a content of the report item, an examination reservation content (examination date and time) of the patient, a pre-examination written consent, an interview result, a written consent, pre-registration, and the like, and records the received patient information in the patient information DB 641. Furthermore, the control unit 66 records observation order information regarding the observation order at the time of observing the organ/site by the endoscope 2 in the observation order information DB 645 based on the operation content input from the laptop personal computer 7 or the like of the operator.

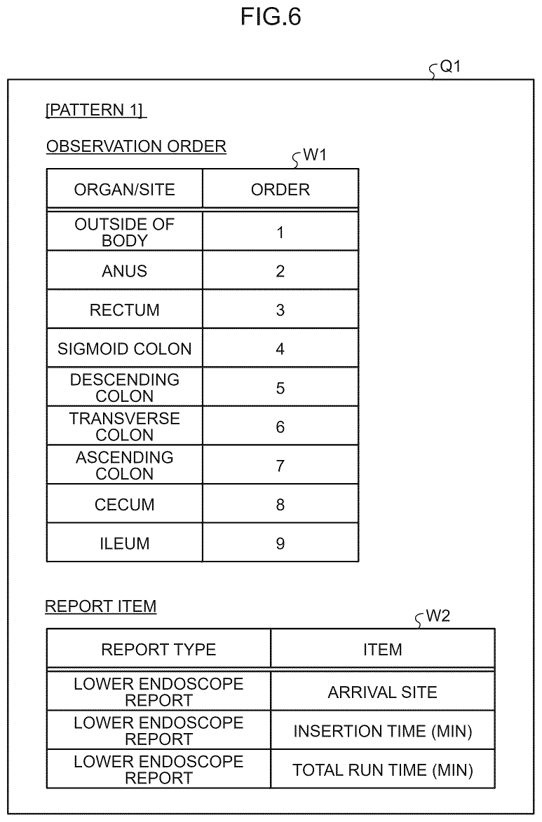

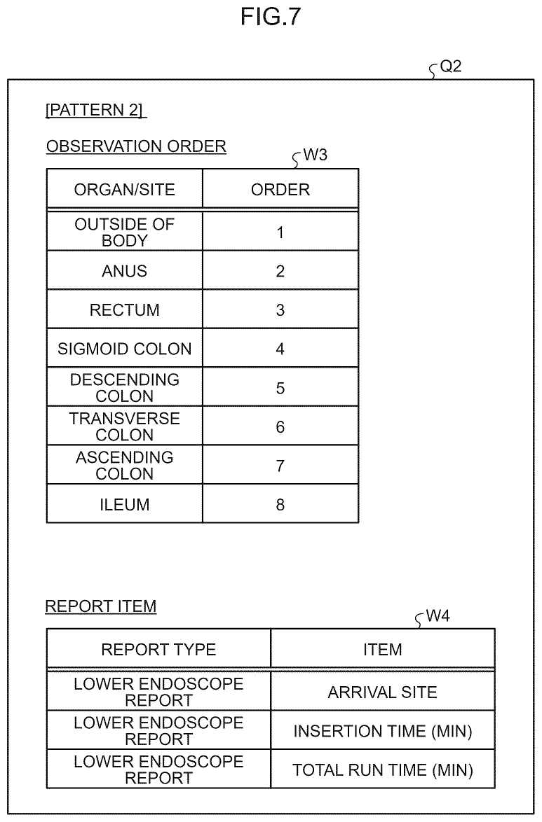

FIG. 6 is a diagram illustrating an example of an observation order of organs/sites and a list of report items. FIG. 7 is a diagram illustrating another example of the observation order of organs/sites and the list of report items.

In organ/site observation order tables W1 and W3 of a pattern Q1 illustrated in FIG. 6 and a pattern Q2 illustrated in FIG. 7, the distal end portion 24 of the insertion portion 21 included in the endoscope 2 (hereinafter, simply referred to as the “distal end portion 24”) is inserted into the body from the outside of the body of the subject, and the order of observation of each organ/site by the endoscope 2 is set for each operator (for each doctor) or for each facility. For example, in the organ/site observation order table W1, the order “9” is set in the organ/site “ileum”.

Similarly, in report item tables W2 and W4, items are set in association with the report type. For example, in the report item table W2, an item “arrival site” is set in a report type “lower endoscope report”. Here, the arrival site is an organ/site of an observation place where the distal end portion 24 can arrive in the subject. In the following description, it is assumed that the pattern Q1 illustrated in FIG. 6 is set.

Referring back to FIG. 5, the description of step S103 and subsequent steps will be continued.

In step S103, the control unit 66 causes the display device 3 or the laptop personal computer 7 to display consistency confirmation and advice between the patient as the subject and the endoscope 2 based on the patient information input in step S102, the past report information recorded in the report information DB 648 associated with the patient information, and the endoscope information recorded in the endoscope DB 642 via the network N100. Specifically, the control unit 66 confirms consistency of setting contents of the endoscope 2 used for observation of the patient based on the patient information, the past report information recorded in the report information DB 648 associated with the patient information, and the endoscope information recorded in the endoscope DB 642. For example, the control unit 66 determines consistency confirmation as to whether the type of the endoscope 2 is suitable for observation according to the observation order at the time of observation of the organ/site set in the endoscope 2 used for observation of the patient, the type of the report item, and the examination reservation content. In this case, the control unit 66 displays a warning on the display device 3 or the laptop personal computer 7 when determining that the type or setting content of the endoscope 2 unsuitable for patient observation is unsuitable. As a result, the operator or the like can recognize that the endoscope 2 connected to the control device 4 is not suitable for observation of the patient.

In addition, based on the patient information, the past report information recorded in the report information DB 648 associated with the patient information, and the endoscope information recorded in the endoscope DB 642, the control unit 66 causes the display device 3 or the laptop personal computer 7 to display, as advice, pieces of information such as the presence or absence of current allergy of the patient, findings of the operator at the time of previous observation of the patient, a tumor of the patient at the previous time, and a site used for follow-up observation. As a result, the operator or the like can grasp the contents of previous requests from an operator or a report creator with respect to the patient.

Subsequently, the control unit 66 determines whether imaging of the endoscope 2 with respect to the subject has started from the control device 4 or the endoscope 2 via the network N100 (step S104). Specifically, upon receiving image data or an activation signal indicating that the endoscope 2 is activated from the control device 4 via the network N100, the control unit 66 determines that imaging of the endoscope 2 with respect to the subject has started.

In a case where it is determined by the control unit 66 that imaging of the endoscope 2 with respect to the subject has started (step S104: Yes), the medical support system 1 proceeds to step S105 to be described later. On the other hand, in a case where it is determined by the control unit 66 that imaging of the endoscope 2 with respect to the subject has not started (step S104: No), the medical support system 1 proceeds to step S107 to be described later.

In step S105, the medical support system 1 executes endoscope imaging processing of inserting the endoscope 2 into the subject and observing the same. Note that details of the endoscope imaging processing will be described later. After step S105, the medical support system 1 proceeds to step S106.

Subsequently, the control unit 66 determines whether imaging of the endoscope 2 with respect to the subject has been completed from the control device 4 or the endoscope 2 via the network N100 (step S106). Specifically, upon receiving a stop signal indicating that the reception of the image data is stopped or the endoscope 2 is stopped from the control device 4 via the network N100, the control unit 66 determines that the imaging of the endoscope 2 with respect to the subject is stopped. In a case where it is determined by the control unit 66 that the imaging of the endoscope 2 with respect to the subject is stopped (step S106: Yes), the medical support system 1 proceeds to step S107 to be described later. On the other hand, in a case where it is determined by the control unit 66 that the imaging of the endoscope 2 with respect to the subject is not stopped (step S106: No), the medical support system 1 returns to step S105.

In step S107, the control unit 66 determines whether a report creator such as a doctor or an observer has started report creation by the laptop personal computer 7 via the network N100. Specifically, the control unit 66 determines whether a request signal requesting predetermined patient information and examination information including an image data group by the endoscope 2, still image data, and a detection result by the image diagnosis device 5, associated with the patient information, has been received from the laptop personal computer 7. In a case where it is determined by the control unit 66 that the report creator has started report creation by the laptop personal computer 7 (step S107: Yes), the medical support system 1 proceeds to step S108 to be described later. On the other hand, in a case where it is determined by the control unit 66 that the report creator has not started the report creation using the laptop personal computer 7 (step S107: No), the medical support system 1 proceeds to step S113.

In step S108, the medical support system 1 executes report creation processing of creating a report based on the observation result of the subject. Note that details of the report creation processing will be described later.

Subsequently, the control unit 66 determines whether a creator who creates a report such as a doctor who performs observation has finished creation of the report by the laptop personal computer 7 via the network N100 (step S109). Specifically, the control unit 66 determines whether an end signal for ending the report creation is received from the laptop personal computer 7, and determines that the report creation is ended in a case where the end signal is received, and determines that the report creation is not ended in a case where the end signal is not received. In a case where it is determined by the control unit 66 that the creator who creates the report of the doctor or the like who performs observation has finished the creation of the report by the laptop personal computer 7 (step S109: Yes), the medical support system 1 proceeds to step S110. On the other hand, in a case where it is determined by the control unit 66 that the creator who creates the report of the doctor or the like who performs observation has not finished the creation of the report by the laptop personal computer 7 (step S109: No), the medical support system 1 returns to step S106.

In step S110, the control unit 66 determines whether the laptop personal computer 7 or the control device 4 has ended the connection to the medical support device 6 via the network N100. In a case where it is determined by the control unit 66 that the laptop personal computer 7 or the control device 4 has ended the connection to the medical support device 6 (step S110: Yes), the medical support system 1 proceeds to step S115 to be described later. On the other hand, in a case where it is determined by the control unit 66 that the laptop personal computer 7 or the control device 4 has not ended the connection to the medical support device 6 (step S110: No), the medical support system 1 proceeds to step S111 to be described later.

In step S111, the control unit 66 determines whether the endoscope 2 connected to the control device 4 has been changed based on the endoscope ID for identifying the endoscope 2 input from the control device 4 via the network N100. In a case where it is determined by the control unit 66 that the endoscope 2 connected to the control device 4 has been changed (step S111: Yes), the medical support system 1 proceeds to step S112 to be described later. On the other hand, in a case where it is determined by the control unit 66 that the endoscope 2 connected to the control device 4 has not been changed (step S111: No), the medical support system 1 returns to step S102.

In step S112, the control device 4 switches setting parameters of the endoscope 2 based on the endoscope ID of the connected endoscope 2. Specifically, the control device 4 switches a parameter of image processing, for example, a parameter of white balance based on the endoscope ID of the connected endoscope 2. After step S112, the medical support system 1 returns to step S102.

In step S113, the control unit 66 determines whether the laptop personal computer 7 or the control device 4 has ended the connection to the medical support device 6 via the network N100. In a case where it is determined by the control unit 66 that the laptop personal computer 7 or the control device 4 has ended the connection to the medical support device 6 (step S113: Yes), the medical support system 1 proceeds to step S114 to be described later. On the other hand, in a case where it is determined by the control unit 66 that the laptop personal computer 7 or the control device 4 has not ended the connection to the medical support device 6 (step S113: No), the medical support system 1 returns to step S101 to be described later.

In step S114, the medical support system 1 executes unexamined processing in a case where the endoscope 2 has not been inserted into the subject. Details of the unexamined processing will be described later. After step S114, the medical support system 1 proceeds to step S115.

Subsequently, the control unit 66 determines whether to end the observation of the subject by the endoscope 2 or the creation of the report by the laptop personal computer 7 (step S115). In a case where it is determined by the control unit 66 that the observation of the subject by the endoscope 2 or the creation of the report by the laptop personal computer 7 is to be ended (step S115: Yes), the medical support system 1 ends the present processing. On the other hand, in a case where it is determined by the control unit 66 that the observation of the subject by the endoscope 2 or the creation of the report by the laptop personal computer 7 is not to be ended (step S115: No), the medical support system 1 returns to step S102.

### Overview of Endoscope Imaging Processing

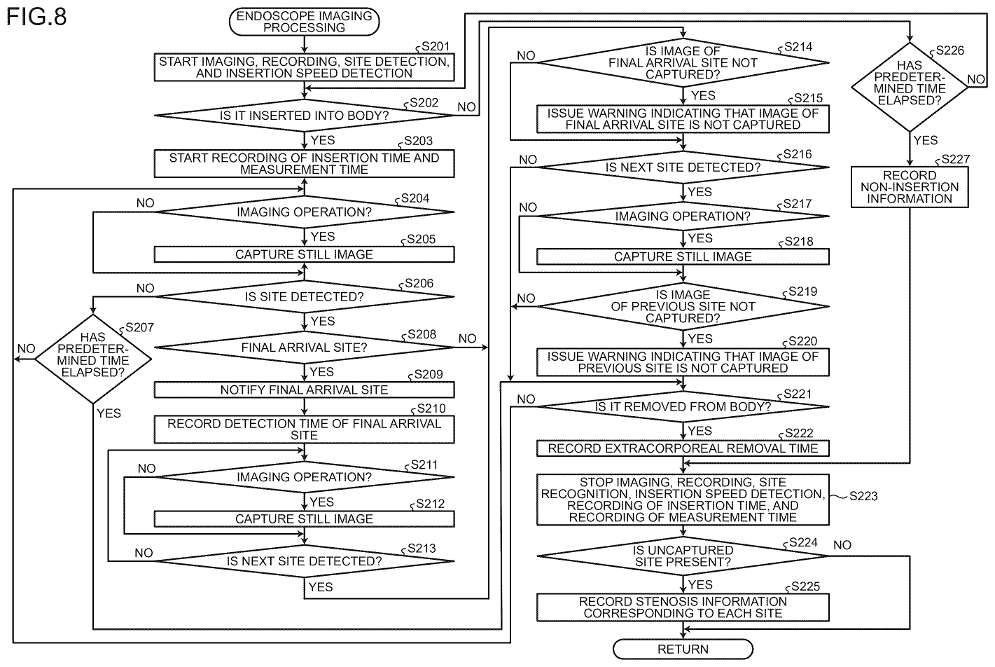

Next, details of the endoscope imaging processing described in step S105 of FIG. 5 will be described. FIG. 8 is a flowchart illustrating an outline of endoscope imaging processing.

As illustrated in FIG. 8, first, the control unit 410 starts imaging of the endoscope 2, recording of image data captured by the endoscope 2, site detection of an organ/site by the image diagnosis device 5, and detection of an insertion speed of the insertion portion 21 of the endoscope 2 by the image diagnosis device 5 (step S201). Specifically, the imaging controller 410a causes the endoscope 2 to start imaging. In this case, the communication controller 410e outputs image data (live view image) sequentially captured and generated by the endoscope 2 to the image diagnosis device 5 in chronological order. At this time, the image diagnosis device 5 starts detection of the organ/site and the characteristic region included in the image data based on the image data input from the control device 4 and the first inference model, and outputs the detection result information to the control device 4. Furthermore, the image diagnosis device 5 starts detection of the insertion speed of the endoscope 2 and counting of the measurement time based on the image data input from the control device 4 and the second inference model, and outputs speed information including the insertion speed information regarding the insertion speed and the measurement time to the control device 4. The control unit 410 generates examination information in which the detection result information and the speed information input from the image diagnosis device 5 are associated with patient information input from the medical support device 6 or the input unit 407, and starts to record the examination information in the examination information recording unit 408c.

Subsequently, the determination unit 410c determines whether the distal end portion 24 of the endoscope 2 has been inserted into the body of the subject based on the detection result of the image diagnosis device 5 (step S202). In a case where the determination unit 410c determines that the distal end portion 24 of the endoscope 2 has been inserted into the body of the subject (step S202: Yes), the control device 4 proceeds to step S203 to be described later. On the other hand, in a case where it is determined by the determination unit 410c that the distal end portion 24 of the endoscope 2 is not inserted into the body of the subject (step S202: No), the control device 4 proceeds to step S225 to be described later.

In step S203, the control unit 410 starts recording an insertion time at which the distal end portion 24 of the endoscope 2 is inserted into the body of the subject and a measurement time of the subject by the endoscope 2 in the examination information recording unit 408c.

Subsequently, the determination unit 410c determines whether the operator has performed an imaging operation on the operating portion 22 (step S204). Specifically, in a case where a release signal is input from the operating portion 22, the determination unit 410c determines that the operator has performed the imaging operation on the operating portion 22. In a case where it is determined by the control unit 410 that the operator has performed the imaging operation on the operating portion 22 (step S204: Yes), the control device 4 proceeds to step S205 to be described later. On the other hand, in a case where it is determined by the determination unit 410c that the operator does not perform the imaging operation on the operating portion 22 (step S204: No), the control device 4 proceeds to step S206 to be described later.

In step S205, the imaging controller 410a causes the imaging element 203 to capture a still image (still image data). Specifically, the imaging controller 410a records, in the image data recording unit 408b, a still image file in which still image data generated by the imaging element 203 is associated with an imaging time at which the imaging element 203 has captured the still image data. At this time, the communication controller 410e outputs the still image file to the medical support device 6 via the network N100. Here, the still image data is an image having a higher resolution (a larger number of pixels) than that of the live view image data (for example, a 4K image). After step S205, the control device 4 proceeds to step S206.

Subsequently, the determination unit 410c determines whether the image diagnosis device 5 has detected a site of the subject (step S206). Specifically, the determination unit 410c determines whether detection result information including organ information obtained by detecting a site of any organ in the subject and a detection time of a detection result obtained by recognizing the site of the organ is received from the image diagnosis device 5, and determines that the image diagnosis device 5 has detected the site of the subject when receiving the detection result information. In a case where it is determined by the determination unit 410c that the image diagnosis device 5 has recognized the site of the subject (step S206: Yes), the control device 4 proceeds to step S208 to be described later. On the other hand, in a case where it is determined by the determination unit 410c that the image diagnosis device 5 does not recognize the site of the subject (step S206: No), the control device 4 proceeds to step S207 to be described later.

In step S207, the determination unit 410c determines whether a predetermined time has elapsed from the insertion of the endoscope 2 into the subject. In a case where it is determined by the determination unit 410c that the predetermined time has elapsed since the insertion of the endoscope 2 into the subject (step S207: Yes), the control device 4 proceeds to step S221 to be described later. On the other hand, in a case where it is determined by the determination unit 410c that the predetermined time has not elapsed from the insertion of the endoscope 2 into the subject (step S207: No), the control device 4 returns to step S204.

In step S208, the determination unit 410c determines whether the distal end portion 24 of the endoscope 2 has reached a final arrival site based on observation order information of the final arrival site set in the observation order of report creation (refer to, for example, pattern 1 Q1 of FIG. 6) and the site of the subject detected by the image diagnosis device 5. Specifically, in a case where the final arrival site set in the observation order is the ileum, when detecting that the site detected by the image diagnosis device 5 is the ileum, the determination unit 410c determines that the distal end portion 24 of the endoscope 2 has reached the final arrival site, and when the site detected by the image diagnosis device 5 is not the ileum, the determination unit determines that the distal end portion 24 of the endoscope 2 has not reached the final arrival site. In a case where it is determined by the determination unit 410c that the distal end portion 24 of the endoscope 2 has reached the final arrival site (step S208: Yes), the control device 4 proceeds to step S209 to be described later. On the other hand, in a case where it is determined by the determination unit 410c that the distal end portion 24 of the endoscope 2 has not reached the final arrival site (step S208: No), the control device 4 proceeds to step S214 to be described later.

In step S209, the display controller 410d causes the display device 3 to display information indicating that the distal end portion 24 of the endoscope 2 has reached the final arrival site in the subject. For example, the display controller 410d gives a notification to the operator by causing the display device 3 to display information indicating that the distal end portion 24 of the endoscope 2 has reached the “ileum” set as the final arrival site. As a result, the operator can grasp that the distal end portion 24 of the endoscope 2 has reached the final arrival site set in the observation order.

Subsequently, the acquisition unit 410b acquires detection result information of the organ/site recognized by the image diagnosis device 5 and a detection time at which the distal end portion 24 of the endoscope 2 reaches the final arrival site, and records the detection result information and the detection time in the examination information recording unit 408c (step S210). In this case, the acquisition unit 410b records, in the examination information recording unit 408c, the detection result of detecting the final arrival site and the detection time in association with each other.

Thereafter, the determination unit 410c determines whether the operator has performed an imaging operation on the operating portion 22 (step S211). In a case where it is determined by the determination unit 410c that the operator has performed the imaging operation on the operating portion 22 (step S211: Yes), the control device 4 proceeds to step S212 to be described later. On the other hand, in a case where it is determined by the determination unit 410c that the operator has not performed the imaging operation on the operating portion 22 (step S211: No), the control device 4 proceeds to step S213 to be described later.

In step S212, the imaging controller 410a causes the imaging element 203 to capture a still image (still image data). Specifically, the imaging controller 410a records, in the image data recording unit 408b, a still image file in which still image data generated by the imaging element 203 is associated with an imaging time at which the imaging element 203 has captured the still image. At this time, the communication controller 410e outputs the still image file to the medical support device 6 via the network N100. After step S212, the control device 4 proceeds to step S213.

In step S213, the determination unit 410c determines whether the image diagnosis device 5 has detected the next site in the subject. Specifically, the determination unit 410c determines whether detection result information of detecting the next site in the subject is received from the image diagnosis device 5, and determines that the next site in the subject is detected when the detection result information is received. In a case where it is determined by the determination unit 410c that the image diagnosis device 5 has detected the next site in the subject (step S213: Yes), the control device 4 proceeds to step S214 to be described later. On the other hand, in a case where it is determined by the determination unit 410c that the image diagnosis device 5 has not detected the next site in the subject (step S213: No), the control device 4 returns to step S211.

In step S214, the determination unit 410c determines whether an image of the final arrival site has not been captured. Specifically, the determination unit 410c determines whether the still image data obtained by capturing an image of the final arrival site of the organ/site set in the observation order (insertion order) in the report item in the examination information recording unit 408c is recorded in the image data recording unit 408b. More specifically, based on the imaging time of the still image data recorded in the image data recording unit 408b, the determination unit 410c determines whether still image data captured within a predetermined time, for example, within 3 minutes from the detection time of the detection result information of detecting the ileum set as the final arrival site recorded in the examination information recording unit 408c is recorded in the image data recording unit 408b. In a case where it is determined by the determination unit 410c that the image of the final arrival site is not captured (step S214: Yes), the control device 4 proceeds to step S215 to be described later. On the other hand, in a case where it is determined by the determination unit 410c that the image of the final arrival site is captured (step S214: No), the control device 4 proceeds to step S216 to be described later.

In step S215, the display controller 410d causes the display device 3 to display a warning indicating that the image of the final arrival site has not been captured. As a result, the operator can grasp that the image of the final arrival site with respect to the subject has not been captured.

Subsequently, the determination unit 410c determines whether the image diagnosis device 5 has detected the next site in the subject (step S216). Specifically, the determination unit 410c determines whether detection result information including organ information obtained by detecting the next site of the organ in the subject and a detection time of a detection result obtained by recognizing the site of the organ is received from the image diagnosis device 5, and determines that the image diagnosis device 5 has detected the next site of the subject when receiving the detection result information. In a case where it is determined by the determination unit 410c that the image diagnosis device 5 has detected the next site in the subject (step S216: Yes), the control device 4 proceeds to step S217 to be described later. On the other hand, in a case where it is determined by the determination unit 410c that the image diagnosis device 5 has not detected the next site in the subject (step S216: No), the control device 4 proceeds to step S221.

In step S217, the determination unit 410c determines whether the operator has performed an imaging operation on the operating portion 22. In a case where it is determined by the control unit 410 that the operator has performed the imaging operation on the operating portion 22 (step S217: Yes), the control device 4 proceeds to step S218 to be described later. On the other hand, in a case where it is determined by the determination unit 410c that the operator does not perform the imaging operation on the operating portion 22 (step S217: No), the control device 4 proceeds to step S219 to be described later.

In step S218, the imaging controller 410a causes the imaging element 203 to capture a still image (still image data). Specifically, the imaging controller 410a records, in the image data recording unit 408b, a still image file in which still image data generated by the imaging element 203 is associated with an imaging time at which the imaging element 203 has captured the still image. At this time, the communication controller 410e outputs the still image file to the medical support device 6 via the network N100. After step S218, the control device 4 proceeds to step S219.

In step S219, the determination unit 410c determines whether the previously recognized site by the image diagnosis device 5 has not been captured. Specifically, on the basis of the imaging time of each still image data recorded in the image data recording unit 408b, the determination unit 410c determines whether still image data captured within a predetermined time, for example, within 3 minutes from the detection time of the previously recognized site by the image diagnosis device 5 is recorded in the image data recording unit 408b immediately after the image diagnosis device 5 recognizes a new site, which is an organ/site set in the examination information recording unit 408c in the observation order (insertion order) in the report item, by allowing the distal end portion 24 of the endoscope 2 to move into the subject. For example, in a case where the image diagnosis device 5 recognizes the organ/site as an ascending colon, and the still image data obtained by capturing an image of the cecum is not recorded in the image data recording unit 408b although the examination information including the detection result recognized as the cecum by the image diagnosis device 5 is recorded in the examination information recording unit 408c, the determination unit 410c determines whether the image of the previously recognized site by the image diagnosis device 5 is not captured. In a case where it is determined by the determination unit 410c that the image of the previously recognized site by the image diagnosis device 5 has not been captured (step S219: Yes), the control device 4 proceeds to step S220 to be described later. On the other hand, in a case where it is determined by the determination unit 410c that the image of the previously recognized site by the image diagnosis device 5 is captured (step S219: No), the control device 4 proceeds to step S221 to be described later.

In step S220, immediately after the image diagnosis device 5 recognizes a new site by allowing the distal end portion 24 of the endoscope 2 to move into the subject, the display controller 410d causes the display device 3 to display a warning indicating that the image of the previously recognized site by the image diagnosis device 5 has not been captured. As a result, the operator can prevent forgetting to perform imaging due to the movement of the distal end portion 24 of the endoscope 2. After step S220, the control device 4 proceeds to step S221 to be described later.

In step S221, the determination unit 410c determines whether the distal end portion 24 of the endoscope 2 has been removed from the body of the subject based on the detection result from the image diagnosis device 5. In a case where the determination unit 410c determines that the distal end portion 24 of the endoscope 2 has been removed from the body of the subject (step S221: Yes), the control device 4 proceeds to step S222 to be described later. On the other hand, in a case where the determination unit 410c determines that the distal end portion 24 of the endoscope 2 has not been removed from the body of the subject (step S221: No), the control device 4 returns to step S204 to be described above.

In step S222, the control unit 410 records, in the examination information recording unit 408c, an extracorporeal removal time at which the distal end portion 24 of the endoscope 2 is removed from the body of the subject as the examination information based on the detection result from the image diagnosis device 5.

Subsequently, the control unit 410 stops imaging of the endoscope 2, recording of image data captured by the endoscope 2, site recognition by the image diagnosis device 5, and detection of the insertion speed of the insertion portion 21 of the endoscope 2 by the image diagnosis device 5, recording of the insertion time, and recording of the measurement time (step S223).

Subsequently, the determination unit 410c determines whether there is an uncaptured site in any of the organs/sites set in the observation order (insertion order) in the report item in the examination information recording unit 408c based on the examination information recorded in the examination information recording unit 408c (step S224). In a case where it is determined by the determination unit 410c that there is an uncaptured site in any of the organs and sites set in the observation order (insertion order) in the report item in the examination information recording unit 408c based on the examination information recorded in the examination information recording unit 408c (step S224: Yes), the control device 4 proceeds to step S225 to be described later. On the other hand, in a case where it is determined by the determination unit 410c that there is no uncaptured site in any of the organs and sites set in the observation order (insertion order) in the report item in the examination information recording unit 408c based on the examination information recorded in the examination information recording unit 408c (step S224: No), the control device 4 ends the present processing and returns to the main routine in FIG. 4.

In step S225, the control unit 410 adds stenosis information corresponding to each site to the examination information and records the examination information in the examination information recording unit 408c. Specifically, in a case where there is an uncaptured organ/site in the organ/site set in the observation order (insertion order) in the report item in the examination information recording unit 408c, and the final arrival site has not been reached, the control unit 410 adds stenosis information indicating that stenosis has occurred to the organ/site in which the image of the still image data has been first captured, and records the stenosis information in the examination information recording unit 408c. After step S225, the control device 4 ends the present processing and returns to the main routine in FIG. 4.

In step S226, the determination unit 410c determines whether a predetermined time has elapsed without the endoscope 2 being inserted into the subject. In a case where the determination unit 410c determines that the predetermined time has elapsed without the endoscope 2 being inserted into the subject (step S226: Yes), the control device 4 proceeds to step S227 to be described later. On the other hand, in a case where the determination unit 410c determines that the predetermined time has not elapsed without the endoscope 2 being inserted into the subject (step S226: No), the control device 4 returns to step S202.

In step S227, the control unit 410 records non-insertion information indicating that the endoscope 2 is not inserted into the subject in the examination information recording unit 408c as the examination information. Here, the non-insertion information includes a case in which the endoscope 2 is not inserted due to an allergy or the like of the subject, a case in which an abnormality occurs in the endoscope 2 such that the endoscope 2 cannot be inserted into the subject, and a case in which the current endoscope 2 is not assumed to be used for the subject. After step S227, the control device 4 ends the present processing and returns to the main routine in FIG. 4.

### Details of Report Creation Processing

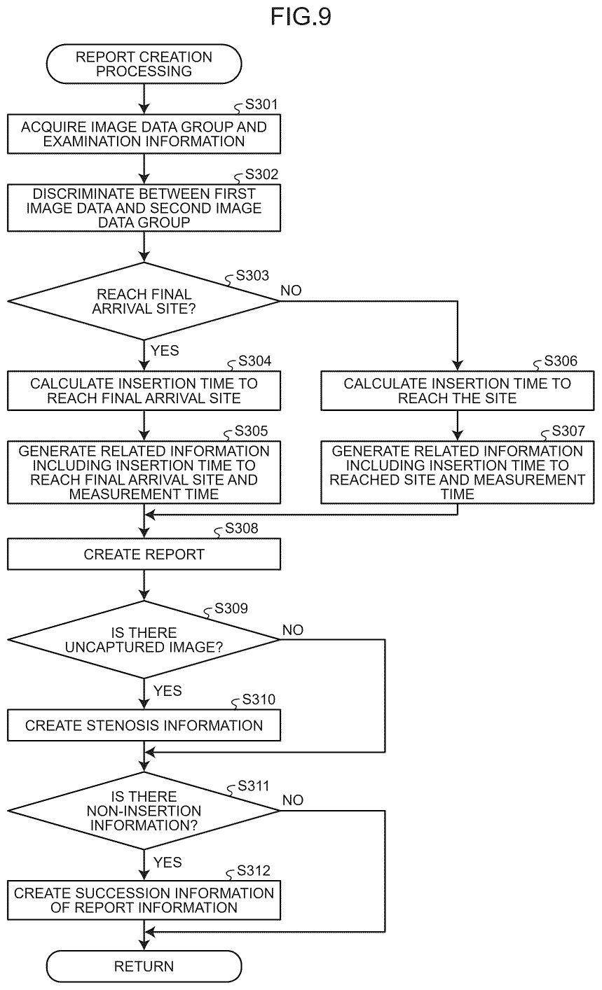

Next, details of the report creation processing in step S108 of FIG. 5 will be described. FIG. 9 is a flowchart illustrating an outline of report creation processing.

As illustrated in FIG. 9, first, the acquisition unit 661 acquires an image data group and examination information from the control device 4 via the network N100 (step S301).

Based on the image data group and the examination information acquired by the acquisition unit 661, the discrimination unit 662 discriminates between first image data captured at the time of observing a predetermined observed region and a second image data group captured after observing the predetermined observed region (step S302).

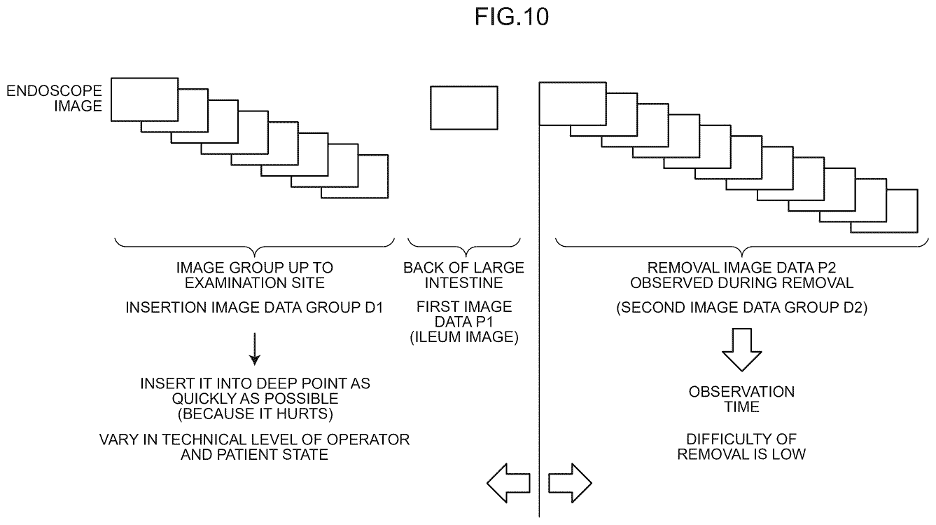

FIG. 10 is a diagram schematically illustrating an outline when the discrimination unit 662 discriminates the image data group into the first image data and the second image data group.

In a case in which the distal end portion 24 of the endoscope 2 (lower endoscope) is inserted into the body from the outside of the body of the subject, the operator inserts the distal end portion 24 into the ileum, which is the final arrival site of the subject, as quickly as possible in order to alleviate the pain on the subject. Furthermore, an insertion time of the distal end portion 24 of the endoscope 2 into the subject as a patient varies depending on the technical level of the operator and the state of the patient. Therefore, as illustrated in FIG. 10, the endoscope 2 captures a large amount of insertion image data group D1 before reaching an examination site, which is the final arrival site.

Subsequently, the operator finishes the insertion of the distal end portion 24 of the endoscope 2 after the distal end portion 24 of the endoscope 2 reaches the final arrival site (ileum) of the subject. In this case, the endoscope 2 generates first image data P1 by capturing an image of the final arrival site. At this time, the operator may operate the operating portion 22 of the endoscope 2 to cause the endoscope 2 to capture an image of the final arrival site, thereby generating still image data.

Thereafter, the operator observes the inside of the subject while removing the distal end portion 24 of the endoscope 2. At this time, the endoscope 2 generates a removal image data group P2 (a second image data group D2). For this reason, the image data group by the conventional endoscope 2 includes the insertion image data group D1 generated by the endoscope 2 when the endoscope 2 is inserted into the subject and the removal image data group P2 (the second image data group D2) generated while the endoscope 2 is removed from the inside of the subject, so that the image data group by the conventional endoscope 2 becomes large in quantity. Conventionally, until reaching the final arrival site of the subject, the operator specifies image data of the final arrival site using a time measurement result using a timer or the like, or specifies image data reaching the final arrival site from an image data group by observing a large number of image data groups after examination of the subject. As a result, it has been difficult for the operator to reduce labor and time required to create the endoscope report.

Therefore, as illustrated in FIG. 10, based on the image data group acquired by the acquisition unit 661 and the examination information, the discrimination unit 662 discriminates between the first image data P1 captured at the time of observing the predetermined observed region and the second image data group D2 captured after observing the predetermined observed region. Specifically, the discrimination unit 662 discriminates a timing at which the insertion state of the distal end portion 24 of the endoscope 2 is switched to the removal state from the image data group based on the image data group and the examination information. For example, as illustrated in FIG. 10, the discrimination unit 662 discriminates, from the image data group including the insertion image data group D1, between the first image data P1 (ileum image) at the back of the large intestine, captured at the time of observing the predetermined observed region, and the second image data group D2 (removal image data group P2) captured after observing the predetermined observed region, thereby discriminating the final arrival site of the subject that the distal end portion 24 of the endoscope 2 has reached. In FIG. 10, the final arrival site is the ileum. However, when a symptom of the subject, for example, stenosis occurs in the ileum, an organ/site immediately before insertion into the ileum becomes the final arrival site.

The discrimination unit 662 discriminates between the first image data and the second image data group by specifying an arrival time at which the distal end portion 24 of the endoscope 2 reaches the final arrival site using any one or more of the following conditions 1 to 3.
The condition 1 is an organ name detected by the image diagnosis device 5, and is an organ name associated in the order set in the order of the organ/site and a detection time at which the organ is detected.
The condition 2 is an imaging time at which still image data associated with still image data is captured.
The condition 3 includes an insertion speed (removal speed) of the distal end portion 24 of the endoscope 2 detected by the image diagnosis device 5 and a measurement time at which the insertion speed is detected.

### In Case Where Conditions 1 to 3 Are Used

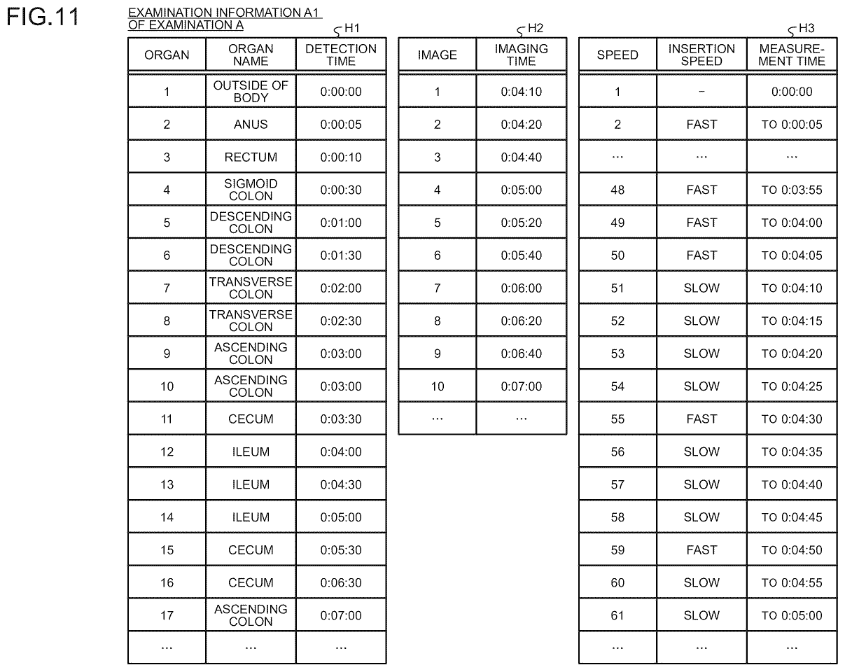

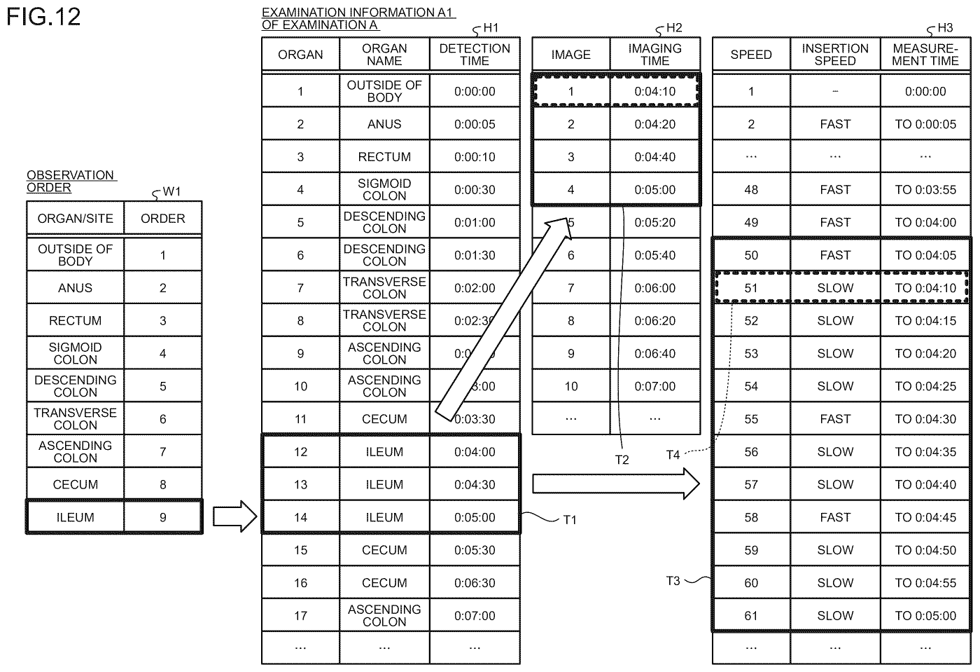

FIG. 11 is a diagram illustrating an example of the examination information acquired by the acquisition unit 661. FIG. 12 is a diagram schematically illustrating a discrimination method in which discrimination is performed by the discrimination unit 662.

Examination information A1 in FIG. 11 includes an organ/observation order table H1 in which organ names that are organ names detected by the image diagnosis device 5 and that are associated in an order set in the observation order of the organ/site and detection times at which the organ is detected are associated with each other, a still image imaging time table H2 in which still image data and an imaging time at which the still image data is captured are associated with each other, and a speed information table H3 in which an insertion speed (removal speed) of the distal end portion 24 of the endoscope 2 detected by the image diagnosis device 5 and a measurement time at which the insertion speed is detected are associated with each other.

As illustrated in FIG. 12, first, the discrimination unit 662 discriminates the organ at the innermost of the distal end portion 24 of the endoscope 2 based on the observation order table H1 and the observation order table W1 (refer to the pattern Q1 in FIG. 6) included in the examination information. In the case illustrated in FIG. 12, the deepest organ/site in an observation order table H1 is the “ileum”, the observation order is “9”, and the detection result by the image diagnosis device 5 includes the “ileum”, so that the discrimination unit 662 discriminates the deepest organ/site as the “ileum”.

Subsequently, the discrimination unit 662 specifies an arrival time at which the distal end portion 24 of the endoscope 2 reaches the final arrival site based on an imaging time T2 of still image data captured within a detection time T1 (0:04:00 to 0:05:00) of the “ileum” determined to be the deepest and a measurement time T3 of the insertion speed. Specifically, the discrimination unit 662 specifies, as the arrival time (0:04:10) at which the distal end portion 24 of the endoscope 2 reaches the final arrival site, a measurement time T4 (0:04:10) which is the imaging time (0:04:10) of the first captured still image data and at which the insertion speed is switched from “fast” to “slow” within the detection time T1 (0:04:00 to 0:05:00) of the “ileum”determined to be the deepest.

As a result, the discrimination unit 662 discriminates, with respect to the image data group, between the first image data (first image data of the ileum at the back of the large intestine) captured at the time of observing the predetermined observed region and the second image data group (second image data group at the time of removal) captured after observing the predetermined observed region based on the specified arrival time.

### In Case Where Conditions 1 and 2 Are Used

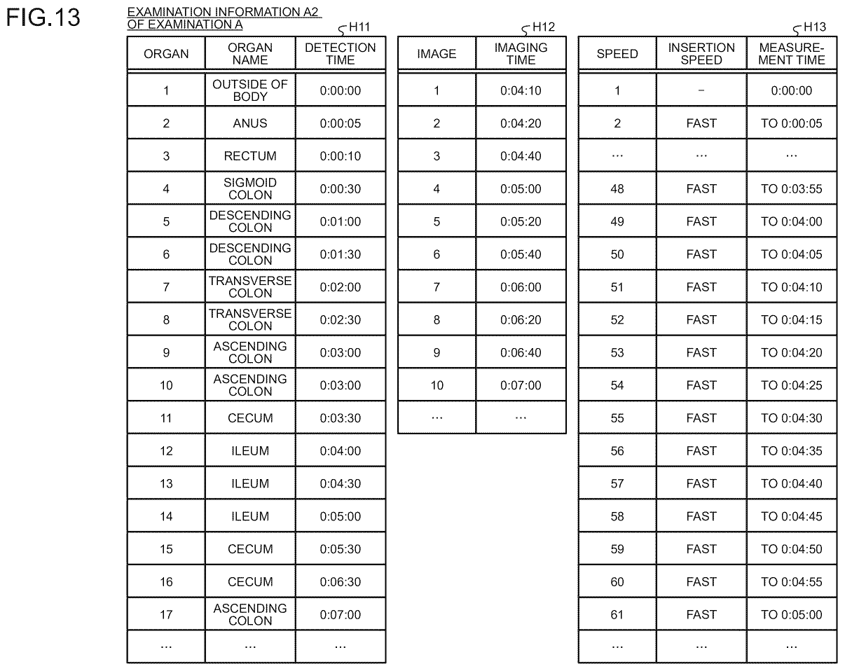

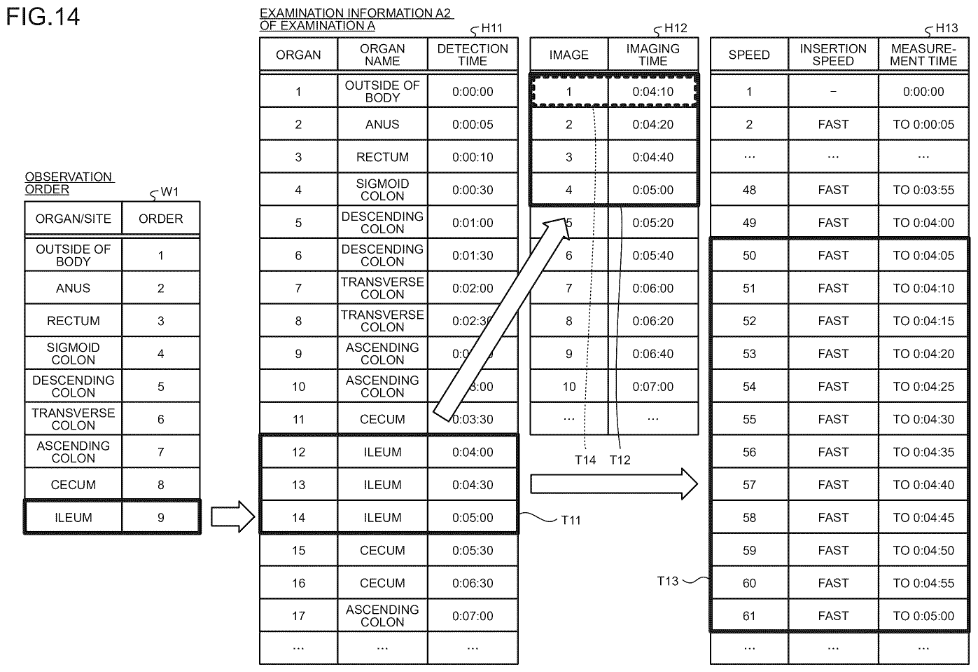

FIG. 13 is a diagram illustrating another example of the examination information acquired by the acquisition unit 661. FIG. 14 is a diagram schematically illustrating a discrimination method in which discrimination is performed by the discrimination unit 662. In FIGS. 13 and 14, a description will be given as to a discrimination method in a case where there is no switching timing of the insertion speed.

Examination information A2 in FIG. 13 includes an organ/observation order table H11 in which organ names that are organ names detected by the image diagnosis device 5 and that are associated in an order set in the observation order of the organ/site and detection times at which the organ is detected are associated with each other, a still image imaging time table H12 in which still image data and an imaging time at which the still image data is captured are associated with each other, and a speed information table H13 in which an insertion speed (removal speed) of the distal end portion 24 of the endoscope 2 detected by the image diagnosis device 5 and a measurement time at which the insertion speed is detected are associated with each other.

As illustrated in FIG. 14, first, the discrimination unit 662 discriminates the organ at the innermost of the distal end portion 24 of the endoscope 2 based on the observation order table H11 and the observation order table W1 (refer to the pattern Q1 in FIG. 6). In the case illustrated in FIG. 14, the deepest organ/site in an observation order table W1 is the “ileum”, the observation order is “9”, and the detection result by the image diagnosis device 5 includes the “ileum”, so that the discrimination unit 662 discriminates the deepest organ/site as the “ileum”.

Subsequently, as illustrated in FIG. 14, since there is no measurement time T13 (0:04:05 to 0:05:00) including a measurement time at which the insertion speed is switched from “fast” to “slow” within a detection time T11 (0:04:00 to 0:05:00) of the “ileum” determined to be the deepest, the discrimination unit 662 specifies an imaging time T14 (0:04:10) of the first captured still image data as an arrival time at which the distal end portion 24 of the endoscope 2 reaches the final arrival site based on an imaging time T12 (0:04:10 to 0:05:00) of the still image data.

As a result, the discrimination unit 662 can discriminate, based on the specified arrival time, between the first image data (first image data of the ileum at the back of the large intestine) captured at the time of observing the predetermined observed region and the second image data group (second image data group at the time of removal) captured after observing the predetermined observed region.

### In Case Where Only Condition 1 is Used

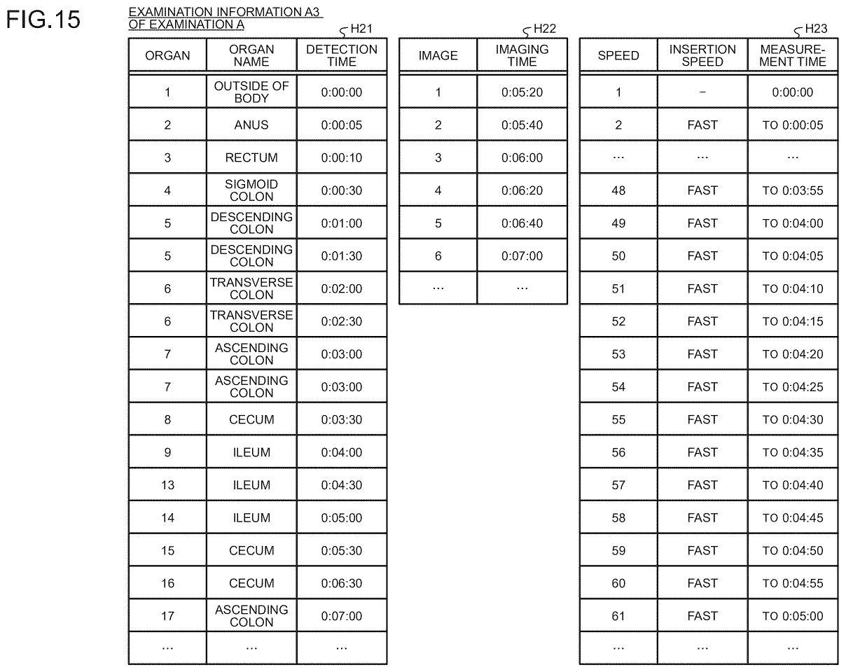

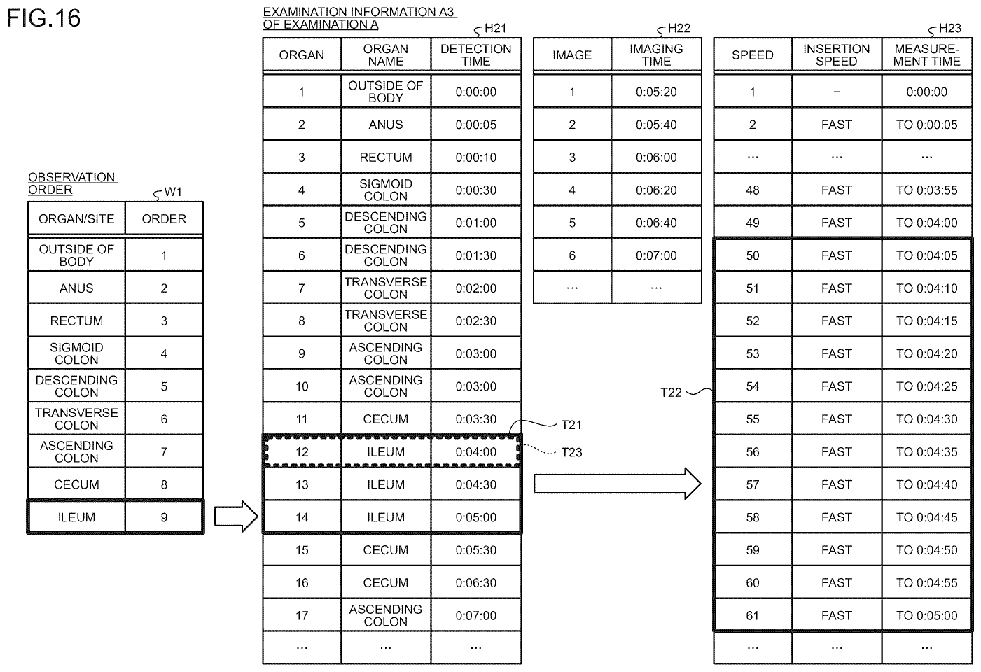

FIG. 15 is a diagram illustrating another example of the examination information acquired by the acquisition unit 661. FIG. 16 is a diagram schematically illustrating a discrimination method in which discrimination is performed by the discrimination unit 662. In FIGS. 15 and 16, a description will be given as to a discrimination method in a case where there is no imaging time of still image data within a detection time T21 of the “ileum” determined to be the deepest.

Examination information A3 in FIG. 15 includes an organ/observation order table H21 in which organ names that are organ names detected by the image diagnosis device 5 and that are associated in an order set in the observation order of the organ/site and detection times at which the organ is detected are associated with each other, a still image imaging time table H22 in which still image data and an imaging time at which the still image data is captured are associated with each other, and a speed information table H23 in which an insertion speed (removal speed) of the distal end portion 24 of the endoscope 2 detected by the image diagnosis device 5 and a measurement time at which the insertion speed is detected are associated with each other.

As illustrated in FIG. 16, since there is no imaging time of still image data within the detection time T21 of the “ileum” determined to be the deepest, and there is no measurement time at which the insertion speed is switched from “fast” to “slow” at a measurement time T22, the discrimination unit 662 discriminates a detection time T23 “0:04:00” of the “ileum” determined to be the deepest as the arrival time at which the distal end portion 24 of the endoscope 2 reaches the final arrival site.

As a result, the discrimination unit 662 can discriminate, based on the arrival time, between the first image data (first image data of the ileum at the back of the large intestine) captured at the time of observing the predetermined observed region and the second image data group (second image data group at the time of removal) captured after observing the predetermined observed region.

In FIGS. 11 to 16, the discrimination unit 662 discriminates the “ileum”, which is the organ/site having the largest numerical value in the order of the organ/site, as the final arrival site by the image diagnosis device 5, but the disclosure is not limited thereto. In a case where there is no organ/site having the largest numerical value in the order of the organ/site, the discrimination unit may discriminate the detection result of the organ/site having the largest numerical value in the order of the organ/site, which is the detection result by the image diagnosis device 5 included in the examination information, as the final arrival site. For example, the discrimination unit 662 discriminates the cecum as the final arrival site when the organ/site having the largest numerical value in the order of the organ/site of the detection result detected by the image diagnosis device 5, for example, the cecum “8” is detected in the examination information.

Referring back to FIG. 9, the description of step S303 and subsequent steps will be continued.

In step S303, the determination unit 663 determines whether the distal end portion 24 of the endoscope 2 has reached the final arrival site (for example, the ileum) preset in the examination item based on an observation order setting division method recorded in the observation order information DB 645 and the examination information. When the determination unit 663 determines that the distal end portion 24 of the endoscope 2 has reached the final arrival site (for example, the ileum) preset in the examination item (step S303: Yes), the medical support device 6 proceeds to step S304 to be described later. On the other hand, when the determination unit 663 determines that the distal end portion 24 of the endoscope 2 has not reached the final arrival site (for example, the ileum) preset in the examination item (step S303: No), the medical support device 6 proceeds to step S306 to be described later.

In step S304, the calculation unit 664 calculates an insertion time until the distal end portion 24 of the endoscope 2 reaches the final arrival site based on the arrival time of the final arrival site discriminated by the discrimination unit 662 and the examination information.

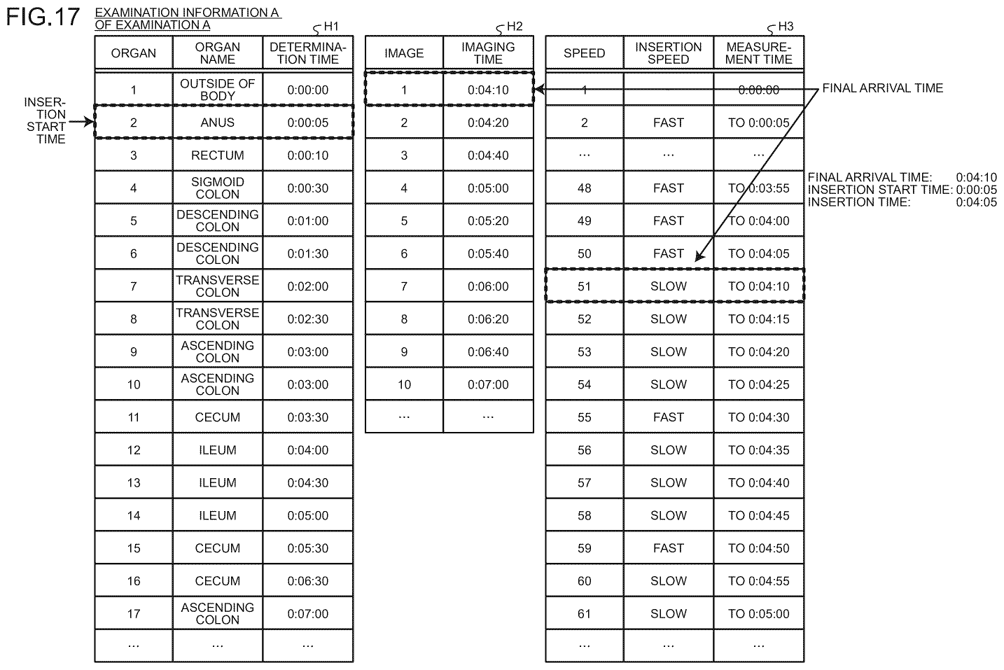

FIG. 17 is a diagram schematically illustrating a method of calculating the insertion time calculated by the calculation unit 664. As illustrated in FIG. 17, the calculation unit 664 sets a detection time (0:00:05) at which the organ is detected as “anus” by the image diagnosis device 5 included in the examination information as an insertion start time at which the distal end portion 24 of the endoscope 2 is inserted into the subject, and calculates an insertion time (0:04:05) based on the insertion start time and a final arrival time (0:04:10) at which it is determined that the final arrival site is reached.

Referring back to FIG. 9, the description of step S305 and subsequent steps will be continued.

The generation unit 665 generates related information including the insertion time until the distal end portion 24 of the endoscope 2 reaches the final arrival site, which is calculated by the calculation unit 664, the insertion start time included in the examination information, and the measurement time of the endoscope 2 based on an outside-of-the-body time at which the outside of the body is detected by the image diagnosis device 5. In this case, the generation unit 665 generates the insertion time, the measurement time, and the insertion speed of the distal end portion 24 of the endoscope 2 by the image diagnosis device 5 as related information related to the movement of the endoscope 2 and records the generated related information in the related information DB 647. After step S305, the medical support device 6 proceeds to step S308 to be described later.

In step S306, the calculation unit 664 calculates an insertion time until the distal end portion 24 of the endoscope 2 reaches the arrival site based on the arrival time to the arrival site discriminated by the discrimination unit 662 and the examination information.

Subsequently, the generation unit 665 generates related information including the insertion time until the distal end portion 24 of the endoscope 2 reaches the arrival site, which is calculated by the calculation unit 664, the insertion start time included in the examination information, and the observation time (measurement time) of the endoscope 2 based on the outside-of-the-body time at which the outside of the body is detected by the image diagnosis device 5 (step S307). In this case, the generation unit 665 generates the insertion time, the measurement time, and the insertion speed of the distal end portion 24 of the endoscope 2 by the image diagnosis device 5 as related information related to the movement of the endoscope 2 and records the generated related information in the related information DB 647. After step S307, the medical support device 6 proceeds to step S308 to be described later.

Subsequently, the creation unit 666 creates report information based on the operation information from the input unit 63 or the laptop personal computer 7, the image data group and the examination information acquired by the acquisition unit 661, the patient information, and the related information generated by the generation unit 665 (step S308). Specifically, the creation unit 666 creates report information (examination report) to which related information regarding the movement of the endoscope 2 including the final arrival site of the distal end portion 24 of the endoscope 2, the insertion time of the distal end portion 24 of the endoscope 2, and the measurement time by the endoscope 2 is added, and records the report information in the report information DB 648.

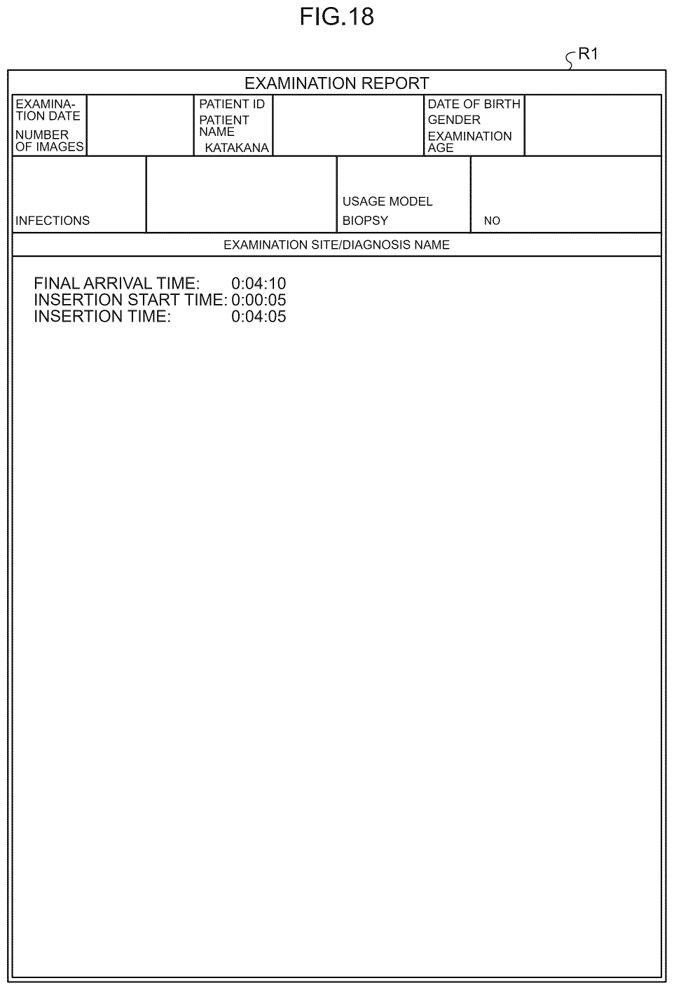

FIG. 18 is a diagram illustrating an example of the examination report created by the creation unit 666. In FIG. 18, a still image captured at the time of observing the subject, findings based on the observation result by the operator, and the like are input to the examination report R1, but detailed description is omitted.

As illustrated in FIG. 18, the creation unit 666 creates an examination report R1 in which at least the insertion time of the endoscope 2 is added to the patient information according to the operation information from the input unit 63 or the laptop personal computer 7. As a result, since the examination report R1 to which the insertion time measured by the operator in the related art is automatically added is created, it is possible to reduce labor and time related to creation of the endoscope report. Note that, although the creation unit 666 adds the final arrival time and the insertion start time to the examination report R1, the disclosure is not limited thereto, and the final arrival site may be automatically added, or still image data at an imaging time close to the detection time of each organ may be automatically added.

Thereafter, the determination unit 663 determines whether there is an uncaptured image in any of the organs/sites set in the report item based on the examination information acquired by the acquisition unit 661 (step S309). When the determination unit 663 determines that there is an uncaptured image in any of the organs/sites set in the report item (step S303: Yes), the medical support device 6 proceeds to step S304 to be described later. On the other hand, when the determination unit 663 determines that there is no uncaptured image in any of the organs/sites set in the report item (step S303: No), the medical support device 6 proceeds to step S305 to be described later.

In step S310, the creation unit 666 creates stenosis information based on the examination information acquired by the acquisition unit 661 and records the stenosis information in association with the related information generated by the generation unit 665.

Subsequently, the determination unit 663 determines whether there is non-insertion information based on the examination information acquired by the acquisition unit 661 (step S311). In a case where it is determined by the determination unit 663 that there is the non-insertion information (step S311: Yes), the medical support device 6 proceeds to step S312 to be described later. On the other hand, when the determination unit 663 determines that there is no non-insertion information (step S311: No), the medical support device 6 returns to the main routine of FIG. 5.

In step S312, the creation unit 666 adds succession information indicating that there is no insertion in the observation order set in the examination information to the report information and creates the report information. After step S312, the medical support device 6 returns to the main routine of FIG. 5.

### Outline of Unexamined Processing

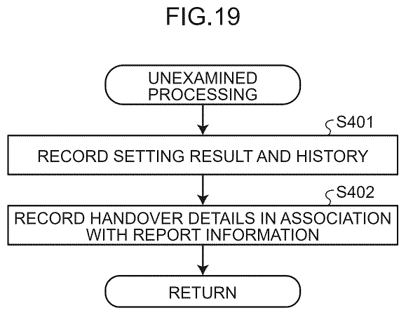

Next, details of the unexamined processing described in step S114 of FIG. 5 will be described. FIG. 19 is a flowchart illustrating an outline of unexamined processing.

As illustrated in FIG. 19, based on a content input from the laptop personal computer 7 or the control device 4, the creation unit 666 records the results of settings, such as the configuration of the subject to be observed and the patient name and a content that has led to non-observation in the patient information DB 641 as a history (step S401). Specifically, examples of the content that has led to non-observation include discontinuation due to a sudden change due to deterioration in physical condition of the subject, discontinuation due to allergic reaction due to prior injection, incompatibility of the type of the endoscope 2 with the subject (patient), lack of maintenance of the endoscope 2, a failure of the endoscope 2, unwashed endoscope 2, and the like.

Subsequently, the creation unit 666 records, in the report information DB 648, the content that has led to non-observation of the endoscope 2 in association with report information of the patient (step S402). As a result, when the operator reads the report information of the patient in the next and subsequent times, it is possible to grasp the content that has led to non-observation. After step S402, the medical support device 6 returns to the main routine of FIG. 5.

According to the embodiment described above, since the generation unit 665 generates the related information related to the movement of the endoscope 2 in the subject in the second image data group based on a discrimination result obtained by allowing the discrimination unit 662 to discriminate between the first image data and the second image data group and the examination information acquired by the acquisition unit 661, it is possible to reduce labor and time for creating the endoscope report.

In addition, according to the embodiment, since the related information is time information when the endoscope 2 captured the second image data group, the time required for the operator to create the endoscope report can be easily grasped, and thus, it is possible to reduce labor and time related to creation of the endoscope report.

In addition, according to one embodiment, since the time information includes the insertion time during which the distal end portion 24 of the endoscope 2 is inserted from the outside of the body of the subject to the preset observed region and the observation time during which the endoscope 2 has observed the inside of the subject, it is possible to easily grasp the time required for the operator to create the endoscope report, so that it is possible to reduce labor and time related to creation of the endoscope report.

In addition, according to one embodiment, since the examination information further includes the speed information in which the insertion speed information related to the insertion speed in the distal end portion 24 of the endoscope 2 moving into the subject based on the image data group is associated with the measurement time from the start time at which the detection of the insertion speed is started to the end time at which the detection of the insertion speed is ended, it is possible to create the endoscope report in consideration of the movement of the distal end portion 24 of the endoscope 2.

Furthermore, according to the embodiment, in a case where the generation unit 665 determines that the distal end portion 24 of the endoscope 2 has reached the final arrival site by the discrimination unit 662, the related information is generated. Therefore, it is possible to discriminate necessary data group from a large amount of image data groups. Accordingly, it is possible to reduce labor and time related to creation of the endoscope report.

Furthermore, according to the embodiment, the discrimination unit 662 specifies, based on the detection result information, the imaging time associated with the still image data, and the speed information, the measurement time at which there is the imaging time associated with the still image data within the determination time of the final arrival site included in the detection result information and the speed of the distal end portion 24 of the endoscope 2 is less than the predetermined threshold as the arrival time of the final arrival site, and thus, it is possible to reduce labor and time related to creation of the endoscope report.

Furthermore, according to the embodiment, since the calculation unit 664 calculates the insertion time of the distal end portion 24 of the endoscope 2 based on the arrival time and the insertion start time of the subject based on the examination information, it is possible to reduce labor and time related to creation of the endoscope report.

Furthermore, according to the embodiment, in a case where the determination unit 663 determines that the distal end portion 24 of the endoscope 2 has not reached the final arrival site, the creation unit 666 records the stenosis information indicating that the stenosis has occurred at the latest observation point included in the detection result information in association with the related information, and thus, it is possible to grasp the symptoms and the like of the patient in advance when the operator takes over the patient at a conference or the like or before performing observation again.

Note that, in the embodiment, the discrimination unit 662 of the medical support device 6 discriminates between the first image data and the second image data group. However, the function of the control unit 66 may be provided in the image diagnosis device 5, and the final arrival site and the insertion time of the distal end portion 24 of the endoscope 2 may be determined based on the image data group sequentially generated by the endoscope 2.

Various embodiments can be formed by appropriately combining a plurality of components disclosed in the medical support system according to the above-described embodiment of the present disclosure. For example, some components may be deleted from all the components described in the medical support system according to the above-described embodiment of the present disclosure. Furthermore, the components described in the medical support system according to the embodiment of the present disclosure described above may be appropriately combined with each other.

Furthermore, in the medical support system according to one embodiment of the present disclosure, the above-described “unit” can be replaced with “means”, “circuit”, or the like. For example, the control unit can be replaced with a control means or a control circuit.

In addition, a program to be executed by the medical support system according to one embodiment of the present disclosure is provided by being recorded in a computer-readable recording medium such as a CD-ROM, a flexible disk (FD), a CD-R, a digital versatile disk (DVD), a USB medium, or a flash memory as file data in an installable format or an executable format.

Furthermore, the program to be executed by the medical support system according to one embodiment of the present disclosure may be stored on a computer connected to a network such as the Internet and may be provided by being downloaded via the network.

Note that, in the description of the flowcharts in the present specification, the context of processing between steps is clearly indicated using expressions such as “first”, “thereafter”, and “subsequently”, but the order of processing necessary for implementing the embodiments is not uniquely determined by such expressions. That is, the order of processing in the flowcharts described in the present specification can be changed within a range without inconsistency. In addition, the program is not limited to such a program including simple branch processing, and more determination items may be comprehensively determined and branched.

Although some of the embodiments of the present application have been described in detail with reference to the drawings, these are merely examples, and the embodiments can be implemented in other forms to which various modifications and improvements have been made based on the knowledge of those skilled in the art, including the aspects described in the disclosure section.

According to the present disclosure, it is possible to reduce labor and time related to creation of an endoscope report.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical support device comprising a processor comprising hardware, the processor being configured to:
acquire a first image data group and examination information, the first image data group being generated by allowing an endoscope to continuously capture images of a plurality of observed regions in a subject, the examination information including detection result information obtained by detecting each of the plurality of observed regions based on the image data group,
determine, based on observation order information in which an observation order of a preset organ and a preset site is set, the first image data group, and the examination information, whether a distal end portion of an insertion portion provided in the endoscope reaches a final arrival site set in the observation order,
discriminate, based on determining that the distal end portion reaches the final arrival site, between first image data captured before the distal end portion reaches the final arrival site and a second image data group captured after the distal end portion reaches the final arrival, and
generate, based on a determination result obtained by discriminating between the first image data and the second image data group and on the examination information, related information including time information related to movement of the endoscope in the subject in the second image data group.

2. The medical support device according to claim 1, wherein the related information is time information obtained when the endoscope captures the second image data group.

3. The medical support device according to claim 2, wherein the time information includes:
an insertion time during which the distal end portion is inserted into the preset observed regions from an outside of a body of the subject; and
an observation time during which the endoscope observes an inside of the subject.

4. The medical support device according to claim 3, wherein the examination information further includes speed information obtained by associating insertion speed information with a measurement time, the insertion speed information being related to an insertion speed of the distal end portion moving in the subject based on the first image data group, the measurement time being from a start time at which a detection of the insertion speed is started to an end time at which the detection of the insertion speed is ended.

5. The medical support device according to claim 4, wherein
the detection result information includes:
a type of the organ or the site; and
a detection time at which the type of the organ or the site is detected, and
the processor is configured to discriminate specify an arrival time at which the distal end portion reaches the final arrival site based on the detection time to discriminate between the first image data and the second image data group.

6. The medical support device according to claim 5, wherein
the examination information includes:
still image data captured by the endoscope when a release signal is input from an operating portion included in the endoscope; and
an imaging time at which the still image data is captured, and
the processor is configured to specify the arrival time based on the detection result information and the imaging time associated with the still image data.

7. The medical support device according to claim 6, wherein the processor is configured to, based on the detection result information, the imaging time associated with the still image data, and the speed information, specify, as the arrival time, a measurement time at which there is the imaging time associated with the still image data within a determination time of the final arrival site included in the detection result information and a speed of the distal end portion is below a predetermined threshold.

8. The medical support device according to claim 7, wherein the processor is configured to calculate the insertion time based on the arrival time and an insertion start time with respect to the subject based on the examination information.

9. The medical support device according to claim 8, wherein
the processor is configured to, based on determining that the distal end portion does not reach the final arrival site, generate stenosis information indicating that stenosis occurs at a latest observation portion included in the detection result information and record the stenosis information in association with the related information.

10. The medical support device according to claim 1, wherein the processor is configured to create report information including the related information.

11. A medical support system comprising:
an endoscope;
a control device configured to control the endoscope;
an image diagnosis device; and
a medical support device, wherein
the endoscope includes:
an insertion portion configured to be inserted into a subject; and
a distal end portion provided at a distal end of the insertion portion, the distal end portion being configured to sequentially generate a first image data group by continuously capturing images of a plurality of observed regions preset in the subject and to output the first image data group to the control device,
the image diagnosis device is configured to generate detection result information in which each of the plurality of observed regions is detected for the first image data group using the first image data group and a first inference model that has been pretrained on features of an organ and a site and to output the detection result information to the control device,
the medical support device includes a first processor comprising hardware, and
the first processor is configured to:
acquire, from the control device, the first image data group and examination information including the detection result information generated by the image diagnosis device,
discriminate, based on the first image data group and the examination information, between first image data captured during observation of a predetermined observed region and a second image data group captured after the observation of the predetermined observed region, and
generate, based on a discrimination result obtained by discriminating between the first image data and the second image data group and on the examination information, related information related to movement of the endoscope in the subject in the second image data group.

12. The medical support system according to claim 11, wherein
the control device includes a second processor comprising hardware, and
the second processor is configured to:
determine, based on the detection result information, whether the distal end portion reaches a preset final arrival site of the subject, and
output, based on determining that the distal end portion reaches the final arrival site, information indicating that the distal end portion reaches the final arrival site.

13. The medical support system according to claim 12, wherein
the endoscope further includes an operating portion configured to receive an input of a release signal to instruct imaging, and
the second processor is configured to output, based on the image diagnosis device detecting a next observed region without receiving the input of the release signal from the operating portion after determining that the distal end portion reaches the final arrival site, a warning indicating that an image of the final arrival site is not captured.

14. The medical support system according to claim 13, wherein the second processor is configured to generate and record examination information obtained by associating the detection result information, still image data captured by the endoscope when the release signal is input from the operating portion, and an imaging time at which the still image data is captured with each other.

15. The medical support system according to claim 14, wherein the second processor is configured to, based on determining that the distal end portion does not reach the final arrival site and the release signal is input from the operating portion, add, to the examination information, stenosis information indicating that stenosis occurs in the organ or the site currently detected by the image diagnosis device and record the stenosis information in the examination information.

16. The medical support system according to claim 15, wherein the image diagnosis device is configured to output, to the control device, speed information obtained by associating, using the first image data group and a second inference model that has been trained on an insertion speed of the distal end portion using the first image data group during insertion of the distal end portion into the subject, insertion speed information related to the insertion speed of the distal end portion moving in the subject with a measurement time from a start time at which a detection of the insertion speed is started to an end time at which the detection of the insertion speed is ended.

17. The medical support system according to claim 11, wherein the related information is time information obtained when the endoscope captures an image of the second image data group.

18. The medical support system according to claim 17, wherein the time information includes:
an insertion time during which the distal end portion of the insertion portion provided in the endoscope is inserted into the preset observed region from an outside of a body of the subject; and
an observation time during which the endoscope observes an inside of the subject.

19. A method of operating a medical support device, the method causing the medical support device to execute:
acquiring an image data group and examination information, the image data group being generated by allowing an endoscope to continuously capture images of a plurality of observed regions in a subject, the examination information including detection result information obtained by detecting each of the plurality of observed regions based on the image data group,
discriminating, based on the image data group and the examination information, between first image data captured during observation of a predetermined observed region and a second image data group captured after the observation of the predetermined observed region, and
generating, based on a discrimination result obtained by discriminating between the first image data and the second image data group and on the examination information, related information related to movement of the endoscope in the subject in the second image data group.
